(19) United States
(12) Patent Application Publication
(10) Pub. No.: US 2006/0223058 A1
(43) Pub. Date: Oct. 5, 2006

(54) In vitro association studies

(75) Inventors: David R. Cox, Belmont, CA (US); Brad A. Margus, Boca Raton, FL (US)

Correspondence Address: QUINE INTELLECTUAL PROPERTY LAW GROUP, P.C., P O BOX 458, ALAMEDA, CA, 94501, US

(73) Assignee: Perlegen Sciences, Inc., Mountain View, CA (US)

(21) Appl. No.: 11/373,837

(22) Filed: Mar. 9, 2006

Related U.S. Application Data
(60) Provisional application No. 60/667,903, filed on Apr. 1, 2005.

(51) Int. Cl. C12Q 1/70; C12Q 1/68; G06F 19/00

(52) U.S. Cl. 435/5; 435/6; 702/20

(57) ABSTRACT

Cell and tissue autonomous phenotypes are correlated with genotype information. Correlated genotype information is used to screen individual traits. Methods and systems for correlating cell and tissue autonomous phenotypes to genotype information are provided.

### CROSS REFERENCE TO RELATED APPLICATIONS

This application is related and claims priority to U.S. Ser. No. 60/667,903 “Importance of Deconstructing Complex Traits into Endophenotypes” filed Apr. 1, 2005, which is also incorporated herein by reference for all purposes.

### BACKGROUND OF THE INVENTION

The underlying genetic architecture of a quantitative trait is defined by parameters within or among populations. These parameters include the number of quantitative trait loci (QTL) that affect the trait, the frequencies of alternative polymorphisms at the relevant QTL, the patterns of linkage disequilibrium among the QTL and the magnitude of any effects of the QTL (e.g., additive effects, dominance effects and epistatic effects) on the trait. Understanding which QTL influence a trait, and to what degree, has broad applications in biology, including in molecular medicine (e.g., diagnostics, prognostics, and medical treatment options and outcomes), agriculture (e.g., marker assisted selection (MAS)), and in studies directed towards understanding the biological basis for and evolution of the trait.

Some quantitative phenotypes are the result of one or only a few QTL, making the genetic structure of the phenotype relatively easy to dissect. For example, some phenotypes are driven almost entirely by a single QTL (the single QTL effects estimates for some phenotypic traits are approximately 100%). Where the phenotype is easily determined and the underlying genetic architecture of the phenotype is simple, it is relatively easy to detect the association, i.e., to determine a correlation between a genotype and a phenotype. However, complex traits such as agricultural yield, patient lifespan, response to drug therapy, or clinical drug effects can be the result of numerous interacting QTL that each make a relatively small contribution to the overall phenotype of interest.

The complexity in determining trait-genotype associations for even seemingly simple traits has been demonstrated. For example, Laurie et al. (2004) “The Genetic Architecture of Response to Long-Term Artificial Selection for Oil Concentration in the Maize Kernel” Genetics 168:2141-2155 describe an association study that involved selection of the maize kernel for the seemingly simple phenotype of altered oil concentration, over a period of more than a century (one of the longest running selection experiments in biology). The association study detected about 50 QTL that contributed to changes in oil concentration over the 100+ year period, accounting for about 50% of the observed variance (suggesting that even more than the 50 identified QTL influence the oil composition phenotype). The individual QTL effect estimates for the identified QTL were small and largely additive.

Detection of a relatively large number of QTL for the trait of interest in this case was possible because of extensive breeding population information that was produced by over a century of selection, with concomitant line maintenance and experimental record keeping. While this experiment proves the power of artificial selection over a trait that is influenced by multiple QTL, each of which have a small effect on an overall quantitative phenotype, it is often not possible to correlate genotype information to traits with such complex genetic architecture by this approach, because population size and breeding information are often insufficient to make such correlations with reasonable confidence. Nevertheless, it is plain that the contribution of multiple small QTL effects are significant in terms of at least some traits—in the oil phenotype experiment described by Laurie et al., the populations changed from a 4.7% oil content at the beginning of the experiment to a 19.3% oil content at the end, among the lines selected for high oil content, and a 1.1% oil content in the lines selected for low oil content.

Similarly, in the case of diagnostic applications, it is often not feasible to perform genetic associations on QTL with small effects on an overall phenotype, because the sample sizes and genotype information required to establish correlations become very large for small effects QTL. Nevertheless, it is logical to infer that small effects QTL can have profound consequences in molecular medicine, in a manner analogous to what has proven to be the case for agricultural traits of interest. Thus, it is possible that current correlation studies miss many, if not most, small effects QTL for phenotypes of interest in both molecular medicine and agriculture.

The present invention overcomes these difficulties by providing methods of assaying for QTL in a high-throughput, highly-sensitive fashion. The methods and systems described herein can be made sensitive to the detection of small effects QTL. This, in turn, provides a basis for medical and agricultural applications that were previously unavailable. These and other features will be more fully appreciated upon review of the following.

### SUMMARY OF THE INVENTION

The present invention correlates cell or tissue phenotypes to genotype information. These cell or tissue phenotypes, e.g., responses controlled by cell or tissue autonomous gene expression, are correlated to traits of an individual. Cell and/or tissue phenotypes are easily assayed in a high-throughput fashion, making detection of genotype correlations to the phenotype relatively easy to detect. That is, small effects QTL can be detected because cell and tissue phenotypes (e.g., measured in cell or tissue cultures) can be assayed in a replicate fashion and examined in replicate for genotype correlations, improving the confidence value for any correlations that are observed. Once genotype correlations to cell or tissue phenotypes are observed, the genotype information can be used as a marker for a trait of an individual that correlates with the cell or tissue phenotype(s).

Thus, in a first aspect, the invention provides methods of correlating a cell or tissue autonomous phenotype to a genotype. The methods include detecting variance in a cell or tissue autonomous phenotype in a population of cells or tissues and accessing genotype information for the cells or tissues in the population. The variance is correlated to the genotype information, thereby correlating the cell or tissue autonomous phenotype and the genotype.

The cell or tissue autonomous phenotype is optionally a cellular response to an external stimulus such as radiation, light, heat, chemical substances (e.g., toxins, proteins, lipids, etc.), or the like. The cell or tissue autonomous phenotype can be any cell or tissue autonomous phenotype, including: a radiation response of the cell or tissue; a response of the cell or tissue to an anti-cancer drug; a response of the cell or tissue to a therapeutic agent; a measure of DNA damage repair by the cell or tissue; an immune response by the cell or tissue; an anti-inflammatory response of the cell or tissue; cytokine production by the cell or tissue; rate of DNA replication, transcription or translation; insulin sensitivity/resistance of muscle, fat, or liver cells/tissues; level of insulin secretion from pancreatic β cells; cell migration; energy metabolism of the cell or tissue; oxygen consumption of the cell or tissue; an electrical response of the cell or tissue; a flow of ions across a membrane of the cell or tissue; apoptosis of the cell; an expression level of a housekeeping gene of the cell or tissue; an activity of a housekeeping gene product; and cell cycle regulation of the cell or cells of the tissue. Optionally, the cell autonomous phenotype can be correlated with a physiological status of an individual from which a cell or tissue having the cell or tissue autonomous phenotype is derived.

Variance in the phenotype can be detected, e.g., by detecting natural or systematic variation of the cell or tissue autonomous phenotype in the population of cells or tissues. For example, high and low responder cells or tissues for the phenotype at issue in the population can be detected, with the high responder cells or tissues having a higher response than the low responder cells or tissues to a selected stimulus. For example, high responder cells optionally display a higher enzymatic or other response to a stimulus than low responder cells for a given population. The cells or tissues can be normal cells or tissues (e.g., cell cultures derived from normal cells), or can include abnormal cells (e.g., cancer cell lines). The variation that is detected can be variation between normal cell lines, or abnormal cell lines, or between normal and abnormal cell lines. The cell lines in which variation is detected can be from a single species, genus, family, order, class, phylum or kingdom. For example, the cells can be from human sources, primate sources, mammalian sources, vertebrate sources, animals, etc.

In one example embodiment, high responder cells or tissues are optionally selected from the highest 25% of the population of cells or tissues in the response to the selected stimulus and the low responder cells or tissues are optionally selected from the lowest 25% of the population of cells or tissues in the response to the selected stimulus. However, the precise breakdown of cells or tissues into high and low responders can take any of a variety of different forms, e.g., depending on the data set under consideration, e.g., using any statistical method that separates high and low responders in the data set.

The population of cells or tissues that is evaluated can include an in vitro population of primary or cultured cells or tissues derived from “normal” individuals, i.e., individuals who are not known to possess an organismal phenotype of interest, or can be from case and control populations e.g., including cell lines that are expected to posses a phenotype of interest. The cells or tissues may be from essentially any source(s) that provides sufficient variation in the cellular phenotype for the methods presented herein, e.g., fibroblast cell lines, stem cell lines, available organ cell lines or tissue cultures, muscle cell lines or tissue cultures, blood cell lines, available cell banks, available tissue banks, etc. The population of cells or tissues that is evaluated can optionally include an in vitro population of primary or cultured cells or tissues derived from a patient or population of patients. For example, the cells or tissues under consideration can be stem cells isolated from different patients (or produced from other cells isolated from patients). These cells or tissues can be differentiated or dedifferentiated in vitro, if desired. The cells or tissues of the population can be individual cells, or can be formed within whole or partial tissues or natural or artificial scaffolds or aggregates of cells.

In one convenient embodiment, the cells or tissues are from a cell or tissue bank, e.g., wherein the cells or tissues of the bank are genotyped. For example, the cell or tissue bank can have about 1,000 or more different genotyped cell or tissue lines. The cells or tissues of the bank can be evaluated for one or more cell-autonomous phenotype(s). Similarly, the population of cells or tissues can include a population of cells or tissue samples derived from a population of patients. The cell or tissue banks can include cells derived from “normal” cell sources, or can be known to be derived from abnormal cell sources (e.g., cancer cell lines, etc.). In certain embodiments, cell lines from the cell banks are “normal” and the variation detected in a cell phenotype is simply natural variation in the trait between “normal” cell lines, while in other embodiments, variation can be detected within abnormal cell lines, or between normal and abnormal cell lines. The cell lines from the cell banks can be from same or different organisms (e.g., the cell lines that are tested can be human, or primate, or mammalian, etc.).

Detecting variance for a given phenotype can include separating the population of cells or tissues into case and control sets based upon the variance of the cell or tissue autonomous phenotype in the population. The population of cells or tissues can also include positive control cells or tissues, or negative controls, or both. Optionally, the control(s) and the case set(s) are derived from common cell or tissue lines or types.

Typically, the cell or tissue autonomous phenotype is detected a plurality of times for each tissue or cell type, clone, or line within a population, thereby amplifying correlation certainty during correlation events. For example, the phenotype can be detected 100 or more times for each cell or tissue type. The ability to detect the cell or tissue autonomous phenotype multiple times for any correlation event is a significant advantage over standard correlation associations between phenotypes and genotypes generally, where replicate studies for a given individual may be difficult to perform.

Thus, one advantage of the present invention is the ability to detect small variations in phenotypes in a population and to correlate them to small effects QTL. That is, because correlations can be verified by replicate analysis, relatively small variations in phenotype can be screened for and correlated to genotypes. This makes it possible to detect, e.g., small additive effects of particular polymorphisms that are missed in standard correlation studies. Thus, for example, a portion of the polymorphisms detected optionally display 10% or less phenotypic variation in a patient population.

Accessing genotype information can include identifying polymorphisms (e.g., haplotype information) in members of the population of cells, or more generally, detecting at least one genotype for members of the population. These polymorphisms or genotypes can be detected before or after detection of the phenotype, and the genotype information can generally be accessed before or after phenotype detection. There is no particular limit on the number of polymorphisms that can be detected and screened for correlation to the phenotype, e.g., a genotype can easily include about 100,000 SNPs, 250,000 SNPs, or more. Genotypes can be screened on a genome-wide basis, or on a targeted basis (e.g., where some hypothesis regarding structure-function exists, or is to be tested by testing for a given correlation). Thus, at least a portion of the polymorphisms are optionally pre-selected to have an effect on, or a predicted effect on, the cell autonomous phenotype, or to have a correlation or predicted correlation to the cell autonomous phenotype. Determining polymorphisms can include determining a haplotype pattern for a cell or related individual, a set of genotypes (a “genetic bar code”) or the like that correlates with a given cell autonomous phenotype.

Correlations between the genotype or cell autonomous phenotype and a patient condition can also be determined (or predicted). That is, a standard validation study can be performed to test any hypothesis regarding a relationship between the genotype or cell autonomous phenotype and the patient condition. Once a statistical correlation between the genotype or cell autonomous phenotype and the patient condition is identified, correlations between any of a variety of serious diseases or treatment outcomes and a genotype or phenotype (e.g., correlation to a treatment side effect, a side effect predisposition, disease state, a disease predisposition, a disease prognosis, a treatment response, a treatment efficacy, etc.) can be used to design a diagnostic assay for the condition (by detecting the genotype or cell autonomous phenotype). That is, correlation information can be used for diagnosing, detecting, detecting a predisposition for, predicting an outcome of, selecting a treatment regimen for a disease and/or the like, in a patient, based upon a correlation between a cell autonomous phenotype of the patient and the genotype.

Thus, a related class of methods for diagnosing, detecting, detecting a predisposition for, predicting an outcome of and/or selecting a treatment regimen for a disease are provided by the present invention. These methods include, e.g., detecting at least one in vitro cell or tissue autonomous phenotype of a tissue or cell from a patient (or derived from a patient) and/or detecting a genotype correlated to the autonomous phenotype. A database is accessed that includes a correlation between the cell or tissue autonomous phenotype and/or genotype and one or more of, e.g.: disease, predisposition to the disease, prognosis of the disease, and/or treatment efficacy or response for or to the disease. Based on the correlation, the method includes diagnosing, detecting, detecting a predisposition for, predicting an outcome of and/or selecting a treatment regimen for the disease.

Thus, for example, the cell or tissue autonomous phenotype can be an indicator for the physiological status of the patient. Such phenotypes include: a radiation response of the cell or tissue; a response of the cell or tissue to an anti-cancer drug; a response of the cell or tissue to a therapeutic agent; a measure of DNA damage repair by the cell or tissue; an immune response by the cell or tissue; an anti-inflammatory response of the cell or tissue; cytokine production by the cell or tissue; energy metabolism of the cell or tissue; oxygen consumption of the cell or tissue; rate of DNA replication, transcription or translation; insulin sensitivity/resistance of muscle, fat, or liver cells/tissues; level of insulin secretion from pancreatic β cells; cell migration; an electrical response of the cell or tissue; a flow of ions across a membrane of the cell or tissue; apoptosis of the cell or tissue; an expression level of a housekeeping gene of the cell or tissue; an activity of a housekeeping gene product; and cell cycle regulation. The relevant disease can include, e.g., cancer, an infectious disease, a viral infection, a bacterial infection, an immune disorder, an autoimmune disorder, obesity, diabetes, cardiovascular disease, a metabolic disorder, metabolic syndrome, a neurodegnerative disease, a CNS disorder, a transplant-related condition, and/or a genetic disease.

The treatment regimen for such a disease that is under consideration can be, e.g., surgery, exposure to radiation, administration of a drug, administration of an anti-cancer drug, administration of an anti-viral drug, administration of an antibiotic, administration of an immune suppressor or enhancer, administration of a cardiovascular drug, administration of a cholesterol level regulating drug, administration of a neurological drug, administration of an anti-rejection drug, administration of an enzyme inhibitor, administration of an enzyme activator, diet, and exercise.

A cell or tissue that is genotyped or tested for a cell or tissue response can be, e.g., from a cell or tissue bank, or taken directly from the patient, or derived from the patient or cell bank by culture, differentiation and/or dedifferentiation. A cell or tissue autonomous phenotype for the patient or a cell culture line is optionally verified in replicate experiments using a plurality of cells from the patient or culture. Detecting the genotype optionally includes detecting a set of selected polymorphisms that correlate, positively or negatively, to the cell or tissue autonomous phenotype.

In one aspect, only one or a few polymorphisms need to be screened to detect a particular set of polymorphisms that correlates with a given cell or tissue autonomous phenotype. This makes it possible to provide a simple and convenient diagnostic application for detecting the correlation of interest. For example, the set of selected polymorphisms optionally includes fewer than 100 polymorphisms, or even less than 10 polymorphisms, greatly simplifying detection formats.

Detecting the genotype optionally includes detecting a set of selected polymorphisms that correlate, positively or negatively, to a disease, a predisposition to the disease, a prognosis of the disease, and/or a treatment response or efficacy for the disease. For example, a database of correlations generated by the methods herein can include a lookup table that comprises correlation relationships for the cell autonomous phenotype and/or genotype and the disease, the predisposition to the disease, the prognosis of the disease, and/or the treatment response or efficacy for the disease. The database is optionally a heuristic database that refines the correlation between genotype, cell autonomous phenotype, and/or the disease, predisposition to the disease, prognosis of the disease, and/or treatment response or efficacy for the disease, based upon inputs regarding the correlation. The heuristic database can include a neural network (NN), a statistical model (SM), a hidden Markov model (HMM), principal component analysis (PCA), classification and regression trees (CART), multivariate adaptive regression splines (MARS), genetic algorithms (GA), multiple linear regression (MLR), variable importance for projection (VIP), inverse least squares (ILS), partial least square (PLS) or any other suitable process or statistical framework.

Systems and kits for practicing the methods are also a feature of the invention, e.g., including cell or tissue response assay components, look up tables with correlations between genotype and phenotype, diagnostic components that monitor presence of polymorphisms or cell response phenotypes, instructions for practicing the methods, software for computer implemented aspects, and the like.

### DETAILED DESCRIPTION

### Overview

Variations in phenotype are correlated with genotype information to provide easily detectable genetic markers for phenotypic variation. That is, genetic markers serve as proxies for detecting trait variants, which can be complex, or even difficult to detect (e.g., certain phenotypes manifest themselves only in certain environments or during certain developmental stages). This basic approach enables modern diagnostic medicine, marker assisted selection (MAS) in crop and animal breeding, and many other aspects of modern biology. However, this approach has certain limitations, particularly with respect to the underlying correlation analysis that establishes a correlation between the marker and the trait. For example, small (but potentially significant) phenotypic variations are often not robustly correlated to genotype information in a given population, e.g., because the standard deviation for a given trait as compared to genotype can be large. This necessitates potentially very large breeding populations and sample sizes before relevant correlations can be detected with an appropriate statistical confidence. The present invention overcomes these limitations.

The present invention recognizes that physiological variation in individuals (including humans and agriculturally relevant individuals) is partially a result of variations in processes that take place within the cells and tissues of the individual. That is, individuals in a varied population display different traits, such as disease outcomes, drug responses, drug side effects, etc., because cells and tissues of the individuals are different for different individuals. Rather than simply detecting correlations between genotypic information and phenotypic information in individuals, the present invention initially identifies correlations between cellular or tissue phenotypes (e.g., cell or tissue autonomous responses) and genotypes. Once these relationships are established and validated, the relevant genotypic information can be used as a marker to detect relevant trait information for an individual.

The cellular or tissue phenotypes themselves are also proxies for traits of an individual. That is, a cellular or tissue phenotype can be detected and used as a marker for an overall phenotypic trait of the individual. For example, a radiation response of the cell or tissue can be correlated to a treatment outcome or side-effect involving radiation. Similarly, a drug response of the cell or tissue to an anti-cancer drug can be used as a marker for an individual's drug response phenotype. In many cases, these relationships between cellular or tissue phenotype and traits of an individual will be suggested by the nature of the cellular or tissue phenotype at issue, and standard correlation analysis can be used to verify whether a correlation between the cellular or tissue phenotype and the traits of interest exists. As noted, in those instances where a genotypic correlation to the cellular or tissue phenotype is identified and validated, the (easily detectable) presence of the relevant genetic marker(s) can be used as a proxy for the relevant cellular or tissue phenotype.

Thus, the invention includes at least two overlapping approaches to detecting correlations between cellular or tissue phenotypes and traits of an individual. First, correlations between a trait of an individual and a quantitative cellular or tissue phenotype (or more than one quantitative cell or tissue phenotype) can be directly determined. Second, genotype information can be correlated to quantitative cellular or tissue phenotypes, and can, therefore, also be correlated to traits of an individual (individual traits are also referred to as “organismal phenotypes”). In either aspect, more than one individual trait may correlate with a given quantitative cellular or tissue trait or genotype, or vice-versa. In addition, a first cellular or tissue phenotype or genotype can be used as a marker for a second cellular or tissue phenotype or genotype, where the first and second phenotypes and/or genotypes are correlated (making a single cellular or tissue phenotype assay or genotype assay indicative of potentially multiple cellular or tissue phenotypes, genotypes or individual traits). Any of these approaches are particularly powerful as compared to standard genotype-trait correlation analysis, because cellular or tissue responses can be assayed in high-throughput replicate cell or tissue based assays, to improve the confidence value of any correlations that are observed.

Cell and tissue phenotypes of interest include those regulated by cell and tissue autonomous genes. Some cell and tissue autonomous genes and corresponding cellular processes controlled by such genes are carried out across different cell types, e.g., cellular housekeeping genes may have similar cell-autonomous functions in many different cell or tissue types. Some of these process can include, e.g., a radiation response of the cell or tissue; a response of the cell or tissue to an anti-cancer drug; a response of the cell or tissue to a therapeutic agent; a measure of DNA damage repair by the cell or tissue; an immune response by the cell or tissue; an anti-inflammatory response of the cell or tissue; cytokine production by the cell or tissue; energy metabolism of the cell or tissue; oxygen consumption of the cell or tissue; rate of DNA replication, transcription or translation; insulin sensitivity/resistance of muscle, fat, or liver cells/tissues; level of insulin secretion from pancreatic β cells; production or uptake of certain cell products (e.g., proteins, RNAs, lipids, hormones, growth factors, etc.) or chemicals (e.g., toxins, nutrients, etc.); cell migration; an electrical response of the cell or tissue; a flow of ions across a membrane of the cell or tissue; apoptosis of the cell; an expression level of a housekeeping gene of the cell or tissue; an activity of a housekeeping gene product; cell cycle regulation of the cell or tissue; and many others.

For example, a quantitative cellular or tissue phenotype of interest can be a response to drug exposure, such as the production of a particular enzyme that metabolizes the drug. Responses to the drug may vary in a population of cells or tissues from “hypo response” (decreased enzyme production induced by the drug) to “no response” (no enzyme production detectably induced or inhibited by the drug) to “hyper response” (high enzyme production induced by the drug). The population of cells or tissues at issue can be cell or tissue lines from a plurality of individuals, with each line corresponding to an individual. The response of each cell or tissue line is analyzed relative to the population range of responses and the genotypes of the cell or tissue lines that have the most divergent responses are compared. For example, the cell or tissue lines that have the lowest response (e.g., lowest 25%) can be compared to the cell or tissue lines that have the highest response (e.g., highest 25%). The cells or tissues that are evaluated can correspond to normal individuals, with the variation simply being the natural variation in the population of cells or tissues, or can be variation between normal and abnormal cell or tissue lines, or can be variation observed within abnormal cell or tissue lines.

The cells or tissues can be genotyped prior to any such study and the genotypes stored electronically, so that, after each study, the genotypes for the cell or tissue lines with the most divergent responses are compared without any need for de novo genotyping. Thus, the genotyped lines can be assayed for many different separate phenotypes, in a serial or parallel fashion, with correlations between the genotype and the phenotypes being determined from a single set of genotype information.

The genotypes associated with the response variation can be further analyzed to probe the biological basis for the variation, and/or can be used for diagnostics, prognostics or predictions, e.g., of therapy/treatment responses for a given disorder or disease, e.g., in an individual that comprises the relevant genotype. By detecting all or a subset of relevant genotype information for an individual, one can better evaluate how to treat the individual in a clinical setting.

In some embodiments, stem cell lines can be used for the basic correlation analysis. Stem cells have the potential to differentiate into many different types of cells, so a practitioner can have a single genotype and a single phenotype to be used as a starting point in the assay, regardless of what kind of cell needs to be examined. Stem cells are differentiated into appropriate cell types or tissues for the assays of interest, according to available differentiation methods.

As already noted, replicate assays can be done to bolster the confidence level of the basic correlation analysis. This can include using clonal cells of a single culture in replicate assays, or can include using clonal cells from separate cultures in replicate assays, e.g., to control for cell culture effects. Optionally, many individual cell or tissue lines and their genotypes can be banked and re-assayed whenever a new cell or tissue phenotype assay is to be performed or evaluated. The genotype information can be simple SNPs, or can include any other polymorphism information (insertions, deletions, variation in repeat sequences (LINES, SINES, microsatellites, Alu elements, etc.), translocations, inversions, etc.), and can be selected from (sampled across) the genome. For example, if SNPs are used, the banked genotype information can include all known SNPs, or can include a subset of SNPs that capture a relevant subset of the information from across the genome (e.g., a set of about 250,000 tag SNPs can be used to cover much of the human genome). See, e.g, Hinds, et al. (2005) “Whole-Genome Patterns of Common DNA Variation in Three Human Populations,” Science 307:1072-1079.

### Cell and Tissue Autonomous Phenotypes

A “phenotype” is a trait or collection of traits that is/are observable in a cell, tissue, individual or population. For example, a cell's or tissue's response to a stimulus is an observable phenotype. A “cell autonomous phenotype” is a phenotype displayed by a cell, e.g., of an individual, where the genes or other endogenous cellular components for display of that phenotype are constituents of or produced in the cell. Thus, for example, a mutation in a gene in the cell leads to an altered cell autonomous phenotype of the cell. Similarly, a “cell autonomous gene” is a gene whose activity affects those cells that express it. This is in contrast to a non cell autonomous phenotype, in which the observed phenotype includes or is induced by factors outside of the cell, e.g., cell-signaling molecules that are brought into contact with the cell. In a similar manner, a “tissue autonomous” trait or phenotype is a trait or phenotype of the tissue for which the factors that produce the phenotype are produced by the tissue. A “non-tissue autonomous” phenotype is a trait of the tissue that requires factors from cells outside of the tissue to manifest (e.g., developmental agents or regulatory hormones carried to the tissue, e.g., by the blood stream).

In the present invention, cell or tissue phenotypes (e.g., cell or tissue responses to any of a variety of stimuli) are detected and genetic associations that correlate with the phenotypes are determined. For example, the cells or tissues are grouped by phenotype into high and low responders and genetic differences between the high and low responders are determined. In this context, a “high responder” cell or tissue is a cell or tissue that shows a greater response to a given stimulus (radiation, heat, light, etc.) than a “low responder” cell or tissue. That is, the two terms are relative to each other for the response at issue, within the population under consideration. For example, in a population under consideration, cells or tissues that are in the top 25% for a given response to a given stimulus can optionally be characterized as “high responders” while cells or tissues that are in the bottom 25% can optionally be characterized as low responders. However, the break down of the population into high and low responders can be selected based on any criteria that best matches the data, or the preferences of the practitioner. For example, where the data fits a smooth distribution curve, the high and low responders are selected from appropriate points on the curve. If the data does not fit a smooth, e.g., bell curve, distribution, then the high responders can be selected from the top responders in the data set, with the low responders selected from the bottom responders in the data set based on any relevant statistical model of the data. The variation detected in the cells or tisses need not be in response to a given stimuli, and may instead be variation observed in unstimulated cells or tissues, e.g., oxygen consumption, rate of DNA replication, glucose metabolism, etc.

Cells or tissues that are genotyped or tested for a cell or tissue autonomous condition can be, e.g., from a cell or tissue bank, or can be taken directly from the patient, or relevant agricultural species individual (e.g., a plant, or a livestock animal) or can be derived from a cell line or cell sample from the cell bank, or from the patient, plant or livestock animal by culture, differentiation and/or dedifferentiation of such a cell or tissue. Sources for cell and tissue lines can include any available banks of cell and tissue lines, e.g., the American Type Culture Collection (ATCC, Manassas, Va.); The National Stem Cell Resource (NSCR, Manassas, Va.); the International Cell and Gene Bank (Seoul, South Korea), the Deutsche Sammlung von Mikroorganismen und Zellkulturen (DSMZ, Braunschweig, Germany), the European Collection of Cell Cultures (Salisbury, United Kingdom), the UK stem cell bank (London, UK), cell culture collections searchable through the Common Access to Biological Resources and Information (CABRI) including BCCM, CABI, CBS, CIP, ECACC, DSMZ, ICLC, NCCB and NCIMB, and many others.

A variety of cell culture methods are available and can be used to provide appropriate tissues, cells or culture-derived cell lines. Details regarding cell and tissue isolation and culture procedures can be found, e.g., in Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) Sambrook et al., Molecular Cloning—A Laboratory Manual (3rd Ed.), Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented current) (“Ausubel”)); and Atlas and Parks (eds) The Handbook of Microbiological Media (1993) CRC Press, Boca Raton.

Thus, in one convenient embodiment, the cells or tissues are from a cell or tissue bank, e.g., wherein the cells or tissues of the cell or tissue bank are genotyped. Thousands of cell and tissue banks exist for humans, plants, livestock, etc. Often, some or all of the members of the cell/tissue bank are at least partially identified by genotype. Furthermore, cells and tissues can, of course, be screened for genotype properties, e.g., by detecting hybridization of cellular nucleic acids to genotyping arrays (e.g., arrays designed to detect SNPs).

For example, a cell or tissue bank can have about 1,000 or more different genotyped cell or tissue lines. The cells or tissues of the bank can be evaluated for one or more cell-autonomous or tissue autonomous phenotype(s). Similarly, the population of cells or tissues can include a population of tissue or cell samples derived from a population of patients or other individuals.

A cell or tissue autonomous phenotype for the patient can be correlated with a genotype in replicate experiments using a plurality of clonal cells, e.g., a cell or tissue culture derived from an individual. Replicate cell biology experiments are carried out using standard cell-based assay formats, e.g., in microwell plates (e.g., 96 or 384 well plates), with replicate experiments on a given cell line being conducted, e.g., in parallel on such plates.

Cells and/or tissues can be exposed to any of a variety of stimuli or environmental conditions and cellular responses to the stimuli are detected. These include, for example, exposure to radiation and monitoring of a radiation response; exposure to an anti-cancer drug and monitoring of a response to the anti-cancer drug; exposure to a therapeutic agent and monitoring of a response; exposure of the cell or tissue to a DNA damage causing agent and monitoring DNA damage repair; exposure of the cell or tissue to an immune modulating agent and monitoring of an immune response; exposure of the cell or tissue to an inflammatory agent and monitoring an anti-inflammatory response; exposure of the cell or tissue to a cytokine modulating agent and monitoring cytokine production; exposure of the cell or tissue to an energy source and monitoring energy metabolism (e.g., glucose metabolism); exposing the cell or tissue to oxygen and monitoring oxygen consumption; exposing the cell or tissue to a membrane polarizing or depolarizing agent and monitoring an electrical response of the cell or tissue; exposing the cell or tissue to an ion or ion channel modulator and monitoring a flow of ions across a membrane of the cell or tissue; exposing the cell or tissue to an apoptosis signal and monitoring apoptosis; exposing the cell or tissue to a selected environmental condition and monitoring an expression level or activity of a housekeeping gene; exposing the cell or tissue to a cell-cycle modulating agent or selected environmental condition and monitoring cell cycle regulation of the cell or tissue, exposing the cell to an agent to detect binding or uptake (e.g., insulin, hormones, growth factors, toxins, nutrients, etc.) and/or many others.

Variance in the phenotype can be detected, e.g., by detecting natural or systematic variation of the relevant response phenotype in the population of cells or tissues. For example, high and low responder cells or tissues for the phenotype at issue in the population can be detected, with the high responders having a higher response than the low responders to a selected stimulus. For example, high responder cells optionally display a higher enzymatic or other response to a selected stimulus than low responder cells for a given population. In one example embodiment, high responder cells or tissues are optionally selected from the highest 25% of the population of cells or tissues in the response to the selected stimulus and the low responder cells are optionally selected from the lowest 25% of the population in the response to the selected stimulus. However, the precise breakdown of cells into high and low responders can take any of a variety of different forms, e.g., depending on the data set under consideration, e.g., using any statistical method that separates high and low responders in the data set.

Detecting variance for a given cell or tissue response can include separating the population of cells or tissues into case and control sets based upon the variance of the cell or tissue response in the population set at issue. The population of cells or tissues can also include positive control cells or tissues, or negative controls, or both. Optionally, the positive or negative control cells or tissues and the case control set of cells or tissues are derived from common tissue or cell lines or tissue or cell types.

Optionally, the cell/tissue response(s) can be correlated with a trait (e.g., physiological status) of an individual from which a cell having the cell autonomous phenotype is derived. This is performed, e.g., via standard statistical correlation analysis, in which any hypothesis regarding a cell or tissue phenotype's relationship to a trait of an individual is tested and validated. Once validated, the cell autonomous phenotype can be used as a proxy for a given individual trait, with the correlation between trait and cell response being correlated by correlation analysis, or based upon a biological model that mechanistically relates, e.g., physiological status to the cell response.

### Cell Phonotype Assays

Thousands of cell and tissue based phenotype assays are available. These include assays for radiation response; response to drugs such as anti-cancer drugs; response to therapeutic agents; measures of DNA damage repair by the cell or tissue in response to a stimulus; immune responses; anti-inflammatory responses cytokine production response; energy metabolism; oxygen consumption; rate of DNA replication, transcription or translation; insulin sensitivity/resistance of muscle, fat, or liver cells/tissues; level of insulin secretion from pancreatic β cells; cell migration; electrical response; flow of ions across a membrane of the cell or tissue; apoptosis; expression level monitoring of any gene (e.g., any cell or tissue autonomous gene), including, e.g., housekeeping genes of the cell or tissue; monitoring changes in expression level or activity of any gene product; cell cycle regulation responses; monitoring uptake or secretion of chemicals or biomolecules (proteins, RNAs, lipids, insulin, hormones, growth factors, nutrients, etc.); and many others. These phenotypes may be monitored in response to a particular stimulous, or may be monitored in unstimulated cells.

For example, expression monitoring, in which expression of gene products are monitored can be performed by northern analysis, RT-PCR, microarray-based methods and/or assays that monitor RNA or protein expression levels. Details regarding these methods are found in Sambrook and Ausubel, as well as in, e.g., U.S. patent application Ser. No. 10/845,316, filed May 12, 2004, entitled “Allele-specific Expression Patterns”, and Walker (1998) Protein Protocols on CD-ROM Humana Press, NJ. Cell-based assays can have any of a variety of formats, e.g., optical, chemical, or electrochemical formats. Optical assays can include imaging and non-imaging applications. Imaging assays typically use fluorescence labeling, viewed through a microscope or camera, while flow cytometry is a common example of a non-imaging optical method.

Commercial kits for the detection of relevant gene product activities, such as, for example, enzyme activity, are widely available. For example, the CellProbe^{HT} line of fluorogenic assay kits (96 or 384 well plate-based assays) from Beckman Coulter allows for the measurement of enzyme activity in living cells, and can detect small changes in enzyme activity. Biolog (Hayward Calif.) offers “phenotypic microarrays™” for simultaneously monitoring many different cell phenotypes simultaneously. Cell based assay kits for monitoring drug transport are available, e.g., from Millipore, Inc. (UK), e.g., the “MultiScreen Caco-2” device for monitoring epithelial cell growth and differentiation. A “Human IGF-I TiterZyme EIA kit” is available from Assay Designs (IGF-I has insulin-like effects, and appears to be involved in aging, nutrition, physical activity and cancer). Site-specific histone H3 global methylation assays are available from Epigentek (site-specific methylation of histone H3 plays a role in silencing and activating gene function, and is involved in cancer and other diseases). T cell receptor assays are available from Wisbiomed. The “TransFluor™” assay from Norak (Research Triangle Park, N.C.) monitors agonist-dependent activation and desensitization of G-coupled protein receptors (GCPRs). Biolmage (Denmark), offers an NF-kβ cell-based assay kit. Bioreliance (Rockville, Md.) uses cell based assays to detect carcinogenicity. Guava Technologies, offers detection of Caspase activity in live cells. Tebu Bio (Belgium) offer cell-based kinase and phosphatase assays. Other suppliers of cell based assays include Sigma-Aldrich, Amersham Biosciences (Piscataway, N.J.), and Cellomics (Pittsburg, Pa.). For monitoring cell metabolism, Molecular Devices sells the IonWorks HT, PatchExpress, and OpusExpress electrophysiology systems as well as a line of cell assay systems and instrumentation. CellStat (Palo Alto, Calif.) offers a metabolic activity assay system with a plate well fitted with electrical probes. ACEA Biosciences (San Diego, Calif.) offer the RT-CES cell assay system that employs microelectronic sensors that detect cellular impedance in wells of microtiter plates. Tecan (Männedorf, Switzerland) and many others offer automated culture systems that support cell based assays. Protedyne (Windsor, Conn.) offers the “BioCube” System that integrates automated liquid handling, storage/retrieval, device loading, and plate transfer for cell based assays. Microfluidic platforms for cell based assays are available from Caliper Technologies (MA) and, e.g., through Biotechnology Ireland for The Trinity Centre for Health Sciences (Ireland).

Details Regarding Transmembrane Potential Measurements and Transmembrane Dyes

Many cell based assays include monitoring transmembrane potential (TM potential) and/or flow of dyes across a cell or organelle membrane to track ion channel activity, cell membrane permeability, cell lysis, organelle activity, etc. In general, the distribution of a permeable ion between the inside and outside of a cell or other membrane depends on the transmembrane potential of the cell membrane. In particular, for ions separated by a semi-permeable membrane, the electrochemical potential difference (Δμⱼ) which exists across the membrane, is given by Δμⱼ=2.3 RT log [jₗ]/[j_{o]+zE}_{R}F, where R is the universal gas constant, T is an absolute temperature of the composition, F is Faraday's constant in coulombs, [jₗ] is the concentration of an ion (j) on an internal or intracellular side of the at least one membrane, [jₒ] is the concentration of j on an external or extracellular side of the at least one membrane, z is a valence of j and E_{R} is a measured transmembrane potential. Thus, the calculated equilibrium potential difference (Eⱼ) for ion j=−2.3RT(zF)⁻¹ log [j_{l]/[j}ₒ] (this is often referred to as the “Nernst equation”). See, Selkurt, ed. (1984) Physiology 5^{th} Edition, Chapters 1 and 2, Little, Brown, Boston, Mass. (ISBN 0-316-78038-3); Stryer (1995) Biochemistry 4^{th} edition Chapters 11 and 12, W.H. Freeman and Company, NY (ISBN 0-7167-2009-4); Haugland (1996) Handbook of Fluorescent Probes and Research Chemicals Sixth Edition by Molecular Probes, Inc. (Eugene Oreg.) Chapter 25 (Molecular Probes, 1996) and http://www(dot)probes(dot)com/handbook/sections/2300(dot)html (Chapter 23 of the on-line 1999 version of the Handbook of Fluorescent Probes and Research Chemicals Sixth Edition by Molecular Probes, Inc.) (Molecular Probes, 1999) and Hille (1992) Ionic Channels of Excitable Membranes, second edition, Sinauer Associates Inc. Sunderland, Mass. (ISBN 0-87893-323-9) (Hille), for an introduction to transmembrane potential and the application of the Nernst equation to transmembrane potential. In addition to the Nernst equation, various calculations which factor in the membrane permeability of an ion, as well as Ohm's law, can be used to further refine the model of transmembrane potential difference, such as the “Goldman” or “constant field” equation and Gibbs-Donnan equilibrium. See Selkurt, ed. (1984) Physiology 5^{th} Edition, Chapter 1, Little, Brown, Boston, Mass. (ISBN 0-316-78038-3) and Hille at e.g., chapters 10-13.

Increases and decreases in resting transmembrane potential—referred to as membrane depolarization and hyperpolarization, respectively—play a central role in many physiological processes, including ion-channel gating. Potentiometric optical probes (typically potentiometric dyes) provide a tool for measuring transmembrane potential and changes in transmembrane potential over time (e.g., transmembrane potential responses following the addition of a composition which affects transmembrane potential) in membrane containing structures such as organelles, cells, tissues and in vitro membrane preparations. In conjunction with probe imaging techniques (e.g., visualization of the relevant dyes), dye probes are used to map variations in transmembrane potential across cell and other membranes.

Potentiometric probes include cationic or zwitterionic styryl dyes, cationic rhodamines, anionic oxonols, hybrid oxonols and merocyanine 540. The class of dye determines factors such as accumulation in cells, response mechanism and cell toxicity. See, Molecular Probes 1999 and the reference cited therein; Plasek et al. (1996) “Indicators of Transmembrane potential: a Survey of Different Approaches to Probe Response Analysis.” J Photochem Photobiol; Loew (1994) “Characterization of Potentiometric Membrane Dyes.” Adv Chem Ser 235, 151 (1994); Wu and Cohen (1993) “Fast Multisite Optical Measurement of Transmembrane potential” Fluorescent and Luminescent Probes for Biological Activity, Mason, Ed., pp. 389-404; Loew (1993) “Potentiometric Membrane Dyes.” Fluorescent and Luminescent Probes for Biological Activity, Mason, Ed., pp. 150-160; Smith (1990) “Potential-Sensitive Molecular Probes in Membranes of Bioenergetic Relevance.” Biochim Biophys Acta 1016, 1; Gross and Loew (1989) “Fluorescent Indicators of Transmembrane potential: Microspectrofluorometry and Imaging.” Meth Cell Biol 30, 193; Freedman and Novak (1989) “Optical Measurement of Transmembrane potential in Cells, Organelles, and Vesicles” Meth Enzymol 172, 102 (1989); Wilson and Chused (1985) “Lymphocyte Transmembrane potential and Ca⁺²-Sensitive Potassium Channels Described by Oxonol Dye Fluorescence Measurements” Journal of Cellular Physiology 125:72-81; Epps et al. (1993) “Characterization of the Steady State and Dynamic Fluorescence Properties of the Potential Sensitive dye bis-(1.3-dibutylbarbituric acid) trimethine oxonol (DiBAC₄(3) in model systems and cells” Chemistry of Physics and Lipids 69:137-150, and Tanner et al. (1993) “Flow Cytometric Analysis of Altered Mononuclear Cell Transmembrane potential Induced by Cyclosporin” Cytometry 14:59-69. Classes of cationic membrane permeable dyes that can be used as ion sensing compositions include, e.g., indo-carbocyanine dyes, thio-carbocyanine dyes, oxa-carbocyanine dyes (see Molecular Probes on-line catalogue, updated as of Aug. 10, 2000, at section 23.3, entitled “Slow-Response Dyes;” http://www(dot)probes(dot)com/handbook/sections/2303(dot)html). See also, Sims, et al. (1974) “Studies on the Mechanism by Which Cyanine Dyes Measure Membrane Potential in Red Blood Cells and Phosphatidylcholine Vesicles,” Biochemistry 13, 3315; Cabrini and Verkman (1986) “Potential-Sensitive Response Mechanism of DiS-C3(5) in Biological Membranes,” Membrane Biol 92, 171; Guillet and Kimmich (1981) “DiO-C3-(5) and DiS-C3-(5): Interactions with RBC, Ghosts and Phospholipid Vesicles,” J Membrane Biol 59, 1; Rottenberg and Wu (1998) “Quantitative Assay by Flow Cytometry of the Mitochondrial Membrane Potential in Intact Cells,” Biochim Biophys Acta 1404, 393 (1998). Other useful transmembrane dyes include amino napthylethylenyl pyridinium dyes, and dialkyl amino phenyl polyphenyl pyridinium dyes. The amino napthylethylenyl pyridinium dyes include the ANEP type dyes, e.g., listed in the Molecular Probes catalog (Di-4-ANEPPS, Di-8-ANEPPS, Di-2-ANEPEQ, Di-8-ANEPEQ and Di-12-ANEPEQ). Dialkyl amino phenyl polyphenyl pyridinium dyes include the RH type dyes listed in the Molecular Probes catalog (RH 160, RH237, RH 421, RH 704, RH 414, and RH 461).

In several applications, changes in the level of fluorescence of a labeled cell or tissue sample in the presence of a stimulus are detected, where the change in fluorescence is indicative of a change in transmembrane potential. Typically, these assay methods are useful for detecting the effect of a stimulus (e.g., a drug) on the transmembrane potential of a cell or tissue membrane. Where one is seeking to determine the effect of a drug on a cell's transmembrane potential, e.g., through a change in ion flux, transport, membrane permeability, or the like, one can expose the cell, membrane, etc., to the test compound and the cell or tissue is examined for the presence of a previously absent fluorescent signal (or the absence of a previously present fluorescent signal).

For example, in one assay format, a dye is contacted to a cell or tissue. In accordance with these methods, the cell or tissue can be placed into a reaction vessel, such as a microwell dish, and the level of fluorescence from the composition is measured, optionally over a period of time. This can be used to provide an initial or background level of fluorescence indicative of an existing transmembrane potential for the biological sample. A selected test compound (e.g., drug) is then added to the biological sample (or these procedures are carried out in parallel, providing control and experimental samples). Following this stimulus, the fluorescence level of the biological sample is again measured (typically over time) and compared to the initial fluorescent level or the fluorescence level in a control cell population. Any change in the level of fluorescence not attributable to dilution by the test compound (as determined from an appropriate control) is then attributable to the effect the test compound has on the cell's transmembrane potential.

The assay methods of the present invention are particularly useful in performing high-throughput (greater than 1,000 compounds/day or greater than 1,000 cell types per day, or both) and even ultra-high throughput (e.g., greater than 10,000 compounds and or cells/day). These experiments may be carried out in parallel by a providing a large number of cells or tissues in separate receptacles, typically in a multiwell format, e.g., 96 well, 324 well or 1536 well plates. Test compounds are added to separate wells, and the effect of the compound on the cell or tissue is ascertained, e.g., via the fluorescent signal. These parallelized assays are generally carried out using specialized equipment e.g., as described above to enable simultaneous processing of large numbers of samples, i.e., fluid handling by robotic pipettor systems and fluorescent detection by multiplexed fluorescent multi-well plate readers.

Patch Clamping

As noted above, monitoring of transmembrane dye flow is a preferred method of monitoring test compound effects on ion channels. A second preferred method uses voltage clamping, such as patch clamping.

A voltage clamp allows for the measurement of ion currents flowing across a cell membrane. Originally, the voltage clamp used two electrodes and a feedback circuit for transmembrane measurements. In the original Cole-Marmount voltage clamp, both electrodes are placed inside a cell and transmembrane voltage is recorded through one of the electrodes (the “voltage electrode”) relative to an outside reference (e.g., ground). The second electrode passes current into the cell and is termed the “current electrode”.

Briefly, a “holding voltage” is maintained across the cell membrane. Anytime the cell makes a deviation from this holding voltage by passing an ion current across its membrane, an operational amplifier generates an “error signal”. The error signal is the difference between the holding voltage specified by the experimenter and the actual voltage of the cell. The feedback circuit of the voltage clamp passes current into the cell (via the current electrode) in the polarity needed to reduce the error signal to zero. Thus, the current is applied in a polarity opposite current that the cell is passing across its membrane, and the clamp circuit provides a current that is the mirror image of the cellular current. This mirror or “clamp current” can be easily measured, giving an accurate reproduction of the currents flowing across the cell's membrane (although in the opposite polarity).

A modern variant of this general method is the “patch clamp” which uses a single electrode device. The patch clamp technique is in common use to monitor the flow of ions across a membrane (Neher E (1992) “Nobel lecture. Ion channels for communication between and within cells” Neuron. 8(4):605-12). The patch clamp technique involves applying a very finely drawn glass micropipette onto the surface of a cell to form an electrode. This electrode is pressed against a cell membrane and suction is applied to the inside of the electrode to pull the cell's membrane inside the tip of the electrode. This suction causes the cell to form a tight seal with the electrode (a “giga-ohm seal,” as the electrical resistance of the seal is in excess of one giga-ohm). From this point, at least 4 different experimental approaches can be taken. First, the electrode can be left sealed to a patch of membrane (a “cell-attached patch”). This allows for the recording of currents through single ion channels in that patch of membrane. Second, the electrode can be withdrawn from the cell, ripping a patch of membrane off of the cell. This forms an “inside-out” patch. This is useful when the environment on the inside of an ion channel is to be studied. Third, the electrode can be withdrawn from the cell, allowing a blob of membrane to bud from the cell. When the electrode is pulled away, this blob will part from the cell and reform as a ball of membrane on the end of the electrode, with the outside of the membrane being the surface of the ball (thus the name “outside out patch”). Such “outside out” patching permits examination of the properties of an ion channel when it is protected from the outside environment, but not in contact with it's usual environment. Fourth, the electrode can be left in place, but harder suction is applied to rupture the portion of the cell's membrane that is inside the electrode, providing access to the intracellular space of the cell. This is known as “whole-cell recording”. This method is also sometimes misnamed a “whole cell patch.” The advantage of whole cell recording is that the sum total current that flows across the cell's membrane can be recorded.

Thus, the voltage clamping such as the patch clamp technique allows the recording of single ion-channel currents, or alternatively currents from entire small cells. In the context of the present invention, this provides a platform for the analysis of changes in currents that result from application of a test compound or other stimulus to the cell or tissue of interest.

A modern variant of the classical patch clamp that can be adapted to the present invention is the planar patch clamp, which uses a planar array of PDMS electrodes that mimic a classical glass electrode (Klemic et al. (2002) “Micromolded PDMS Electrode Allows Patch Clamp Electrical Recording From Cells” Biosensors and Bioelectronics 597-604). This modern patch clamp is suited to high throughput patch clamp analysis, allowing many different cells to be analyzed for ion channel activity simultaneously.

Other assays used for monitoring or measuring cell autonomous phenotypes include, but are not limited to, the use of drugs and/or antibodies (e.g., to study cytoskeletal function); immunochemical and cytochemical staining; assays for membrane fluidity (e.g., laser photobleaching, cell fusion, patching and capping, etc.); assays for membrane permeability (e.g., use of liposomes); different microscopic techniques (e.g., light, atomic force, electron, brightfield/darkfield, fluorescence, phase contrast, differential interference contrast, confocal scanning, cryoelectron, polarization, scanning tunneling, etc.); nuclear run-on transcription assay; RNA-DNA hybridization; radioisotopes (e.g., pulse-chase labeling, autoradiography, etc.); centrifugation, use of cDNA probes; immunoblotting; detection of DNaseI sensitivity of active genes in chromatin; colony hybridization; agarose/polyacrylamide gel electrophoresis; chromatography (e.g., thin layer, paper, ion-exchange, gel-filtration, affinity, high-performance liquid, etc.) These methods are well known to those of skill in the art and are more fully described in, e.g., Becker, et al. “The World of the Cell, second edition” © 1986, 1991, The Benjamin/Cummings Publishing Company, Inc.; Alberts et al. “Molecular Biology of the Cell, third edition” ©1983, 1989, 1994, Garland Publishing, Inc.; and Cooper, G. M. “The Cell: A Molecular Approach” ©1997, ASM Press.

### Genotype Information

Accessing genotype information can include identifying polymorphisms (e.g., haplotype information) in members of the relevant population of cells (whether individual cells, cells in culture, or cells in tissues). These polymorphisms or genotypes can be detected before or after detection of the phenotype (e.g., cell or tissue response) and genotype information can generally be accessed before or after phenotype detection. Conveniently, genotyped cell lines are used in cell-based assays to determine cell phenotypes, because genotype information for cells which display phenotypic variation can be used in more than one phenotype assay (to test the same cell lines for more than one phenotype). Similarly, once a correlation is established between a cellular phenotype and a genotype, that cellular phenotype and/or genotype may be used to study many different organismal phenotypes that are believed to be related to the cellular phenotype. For example, a cellular phenotype that is a rate of glucose metabolism may be correlated with genetic loci that, in an organism, may be correlated with diabetes, hypoglycemia, obesity, and the like.

In this context, a “genotype” is a description of one or more polymorphisms of an individual, tissue or cell. That is a “genotype” is the genetic constitution of an individual, tissue or cell, depending on the relevant context, at one or more genetic loci. Genotype is defined by the allele(s) of one or more known loci of the individual/tissue/cell, typically, the compilation of alleles inherited from parental individuals. A “haplotype” in this context is the genotype of an individual/tissue/cell at a plurality of genetic loci on a single DNA strand. Typically, the genetic loci described by a haplotype are physically and genetically linked, (e.g., on the same chromosome strand and less than 50 cM from each other). In this context, a “polymorphism” is a genetic locus that is variable; that is, within a population, the nucleotide sequence at a polymorphism has more than one version or allele. The term “allele” in this context refers to one of two or more different nucleotide sequences that occur or are encoded at a specific locus, or two or more different RNA or polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. One example of a polymorphism is a “single nucleotide polymorphism” (SNP), which is a polymorphism at a single nucleotide position in a genome (the nucleotide at the specified position varies between individuals or populations). An allele “positively” correlates with a trait when it is linked to it and when presence of the allele is an indictor that the trait or trait form will occur in an individual comprising the allele. An allele negatively correlates with a trait when it is linked to it and when presence of the allele is an indicator that a trait or trait form will not occur in an individual comprising the allele.

There is no particular limit on the number of polymorphisms that can be detected and screened for correlation to a phenotype, e.g., a genotype can easily include about 100,000 SNPs, 250,000 SNPs, or more. Genotypes can be screened on a genome-wide basis, or on a targeted basis (e.g., where some hypothesis regarding structure-function exists, or is to be tested by testing for a given correlation). Thus, at least a portion of the polymorphisms are optionally pre-selected to have an effect on, or a predicted effect on the cell or tissue autonomous phenotype, or to have a correlation or predicted correlation to the cell or tissue autonomous phenotype based on, e.g., prior research by the practitioner of the instant invention or others. Determining polymorphisms can include determining a haplotype map for a cell or related individual, a set of polymorphisms or genotypes (a “genetic bar code”), or the like, that correlates with a given cell or tissue autonomous phenotype. For a detailed description on how to determine the risk that an individual will exhibit a particular phenotype using genetic bar codes, see U.S. patent application Ser. No. 10/956,224, filed Sep. 30, 2004, and PCT patent application no. US05/07375, filed Mar. 3, 2005, both of which are entitled “Methods for Genetic Analysis.”

In general, any allele can be screened for correlation to a cell or tissue response. Millions of alleles are known in a variety of relevant organisms, including humans, and agriculturally relevant livestock and crops. One of skill is fully aware of polymorphism databases, including GenBank®, EMBL, DDBJ and the NCBI. Other repositories can easily be identified by searching the internet.

Array-Based Marker Detection

Most typically, cell or tissue lines to be assayed for phenotypic variation are genotyped by querying known alleles, e.g., using nucleic acid arrays that specifically hybridize to particular allelic variants. This is conveniently performed via array-based detection, e.g., as practiced using commercially available arrays, e.g., from Affymetrix (Santa Clara, Calif.) or other manufacturers. Reviews regarding the operation of nucleic acid arrays include Sapolsky et al. (1999) “High-throughput polymorphism screening and genotyping with high-density oligonucleotide arrays” Genetic Analysis: Biomolecular Engineering 14:187-192; Lockhart (1998) “Mutant yeast on drugs” Nature Medicine 4:1235-1236; Fodor (1997) “Genes, Chips and the Human Genome” FASEB Journal 11:A879; Fodor (1997) “Massively Parallel Genomics” Science 277: 393-395; and Chee et al. (1996) “Accessing Genetic Information with High-Density DNA Arrays” Science 274:610-614, and many others. Array based detection is a preferred method for genotyping cell lines of interest, due to the inherently high-throughput nature of array based detection.

A variety of probe arrays have been described in the literature and can be used in the context of the present invention for detection of alleles that can be correlated to the cell or tissue responses noted herein. For example, DNA probe array chips or larger DNA probe array wafers (from which individual chips would otherwise be obtained by breaking up the wafer) are used in one embodiment of the invention. DNA probe array wafers generally comprise glass wafers on which high density arrays of DNA probes (short segments of DNA) have been placed. Each of these wafers can hold, for example, approximately 60 million DNA probes that are used to recognize longer sample DNA sequences (e.g., from individuals or populations, e.g., that comprise markers of interest). The recognition of sample DNA by the set of DNA probes on the glass wafer takes place through DNA hybridization. When a DNA sample hybridizes with an array of DNA probes, the sample binds to those probes that are complementary to the sample DNA sequence. By evaluating to which probes the sample DNA for an individual hybridizes more strongly, it is possible to determine whether a known sequence of nucleic acid is present or not in the sample, thereby determining whether an allele found in the nucleic acid (e.g., from a cell line of interest) is present. One can also use this approach to perform allele specific hybridization (ASH), by controlling the hybridization conditions to permit single nucleotide discrimination, e.g., for SNP identification and for genotyping a sample for one or more SNPs.

The use of DNA probe arrays to obtain allele information typically involves the following general steps: design and manufacture of DNA probe arrays, preparation of the sample, hybridization of sample DNA to the array, detection of hybridization events and data analysis to determine sequence. Preferred wafers are manufactured using a process adapted from semiconductor manufacturing to achieve cost effectiveness and high quality, and are available, e.g., from Affymetrix, Inc of Santa Clara, Calif.

For example, probe arrays can be manufactured by light-directed chemical synthesis processes, which combine solid-phase chemical synthesis with photolithographic fabrication techniques as employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays can be synthesized simultaneously on a large glass wafer. This parallel process enhances reproducibility and helps achieve economies of scale.

Once fabricated, DNA probe arrays can be used to obtain data regarding presence and/or expression levels for genes of interest, e.g., for correlation to cell response data. The DNA samples may be tagged with biotin and/or a fluorescent reporter group by standard biochemical methods. The labeled samples are incubated with an array, and segments of the samples bind, or hybridize, with complementary sequences on the array. The array can be washed and/or stained to produce a hybridization pattern. The array is then scanned and the patterns of hybridization are detected by emission of light from the fluorescent reporter groups. Because the identity and position of each probe on the array is known, the nature of the DNA sequences in the sample applied to the array can be determined. When these arrays are used for genotyping experiments, they can be referred to as genotyping arrays.

The nucleic acid sample to be analyzed is isolated, amplified and, typically, labeled with biotin and/or a fluorescent reporter group. The labeled nucleic acid sample is then incubated with the array using a fluidics station and hybridization oven. The array can be washed and/or stained or counter-stained, as appropriate to the detection method. After hybridization, washing and staining, the array is inserted into a scanner, where patterns of hybridization are detected. The hybridization data are collected as light emitted from the fluorescent reporter groups already incorporated into the labeled nucleic acid, which is now bound to the probe array. Probes that most clearly match the labeled nucleic acid produce stronger signals than those that have mismatches. Since the sequence and position of each probe on the array are known, by complementarity, the identity of the nucleic acid sample applied to the probe array can be identified.

In one embodiment, two (or more) DNA samples may be differentially labeled and hybridized with a single set of the designed genotyping arrays. In this way two (or more) sets of data can be obtained from the same physical arrays. Labels that can be used include, but are not limited to, cychrome, fluorescein, biotin (later stained with phycoerythrin-streptavidin after hybridization), or the like. Two-color labeling is described in U.S. Pat. No. 6,342,355, incorporated herein by reference in its entirety. Each array may be scanned such that the signal from both labels is detected simultaneously, or may be scanned twice to detect each signal separately.

Intensity data is collected by the scanner for all the markers for each of the individuals that are tested for presence of the marker. The measured intensities are a measure indicative of the amount of a particular marker present in the sample for a given individual (expression level and/or number of copies of the allele present in an individual, depending on whether genomic or expressed nucleic acids are analyzed). This can be used to determine whether the individual is homozygous or heterozygous for the marker of interest. The intensity data is processed to provide corresponding marker information for the various intensities. Details regarding the collection and analysis of intensity data are described, e.g., in U.S. Pat. No. 6,586,750; U.S. patent application Ser. No. 10/768,788, filed Jan. 30, 2004, entitled “Apparatus and Methods for Analyzing and Characterizing Nucleic Acid Sequences”; U.S. patent application Ser. No. 10/351,973, filed Jan. 27, 2003, entitled “Apparatus and Methods for Determining Individual Genotypes”; U.S. patent application Ser. No. 10/786,475, filed Feb. 24, 2004, entitled “Analysis Methods for Individual Genotyping”; U.S. patent application Ser. No. 10/970,761, filed Oct. 20, 2004, entitled “Analysis Methods and Apparatus for Individual Genotyping”; and U.S. patent application Ser. No. 11/173,809, filed Jul. 1, 2005, entitled “Algorithm for Estimating Accuracy of Genotype Assignment.”

### Correlating Individual Traits, Cell Autonomous Phenotypes and Genotype Information

The invention optionally includes correlating cellular and/or tissue phenotypes to genotype information and/or to individual traits. Any genotype information that is correlated to a cell or tissue phenotype can also be correlated to a trait of interest in an organism, e.g., once that trait has been correlated to the cell or tissue phenotype. Furthermore, a genotype can also be used as a marker for the cell or tissue autonomous phenotype, e.g., in a validation study that examines whether an association exits between the cell or tissue autonomous phenotype and an individual trait.

Correlations between the genotype and the cell or tissue phenptype can be performed by any method that can identify a relationship between an allele and a cellular/tissue response, or a combination of alleles and a combination of responses. Similarly, any hypothesized association between a cell or tissue phenotype and a trait of an individual can be validated by an association analysis (optionally using the genotype as a marker for the cell or tissue phenotype). Examples of performing association studies to correlate markers and traits are replete in the literature and similar methodology can be applied to the present invention as well. Examples of association studies are found, e.g., in U.S. Pat. No. 6,969,589; U.S. Pat. No. 6,897,025; U.S. patent application Ser. No. 10/448,773, filed May 30, 2002, entitled “Methods for Genomic Analysis”; U.S. patent application Ser. No. 10/286,417, filed May 21, 2002, entitled “Methods for Genomic Analysis”; U.S. patent application Ser. No. 10/426,903, filed Apr. 29, 2002, entitled “Methods for Genomic Analysis”; U.S. patent application Ser. No. 10/227,195, filed Aug. 22, 2002, entitled “Haplotype Structures of Chromosome 21”; U.S. patent application Ser. No. 10/227,152, filed Aug. 22, 2002, entitled “Haplotype Structures of Chromosome 21”; U.S. patent application Ser. No. [unassigned], attorney docket no. 100/1031-10, filed Jan. 31, 2006, entitled “Genetic Basis of Alzheimer's Disease and Diagnosis and Treatment Thereof”; U.S. patent application Ser. No. 10/447,685, filed May 28, 2003, entitled “Liver Related Disease Compositions and Methods”; U.S. patent application Ser. No. 10/427,696, filed Apr. 30, 2003, entitled “Methods for Identifying Matched Groups”; U.S. patent application Ser. No. 10/940,410, filed Sep. 13, 2004, entitled “Methods and Systems for Identifying Predisposition to the Placebo Effect”; U.S. patent application Ser. No. 11/043,689, filed Jan. 24, 2005, entitled “Associations Using Genotypes and Phenotypes”; U.S. provisional patent application No. 60/686,947, filed Jun. 2, 2005, entitled “Parkinson's Disease-Related Disease Compositions and Methods”; PCT patent application no. US05/007375, filed Mar. 3, 2005, entitled “Methods for Genetic Analysis”; PCT patent application no. US05/044876, filed Dec. 8, 2005, entitled “Markers for Metabolic Syndrome Obesity and Insulin Resistance”; U.S. provisional patent application No. 60/722,357, filed Sep. 30, 2005, entitled “Methods and Compositions for Screening and Treatment of Disorders of Blood Glucose Regulation”; U.S. provisional patent application No. 60/722,636, filed Sep. 30, 2005, entitled “Methods and Compositions for Screening and Treatment with Classes of Drugs”; U.S. provisional patent application No. 60/740,971, filed Nov. 29, 2005, entitled “Markers for Breast Cancer”; U.S. provisional patent application No. 60/713,879, filed Sep. 2, 2005, entitled “Modulation of Skin Color”; U.S. provisional patent application No. 60/721,835, filed Sep. 28, 2005, entitled “Rheumatoid Arthritis Association Study”; and U.S. provisional patent application No. [unassigned], attorney docket no. 300/1083-00, filed Jan. 19, 2006, entitled “Markers for Myocardial Infarction.”

Thus, for example, marker polymorphisms or alleles are “correlated” with a specified cell or tissue phenotype when they can be statistically linked (positively or negatively) to the phenotype. This correlation may be causal in nature, but it need not be—simple genetic linkage to (association with) a locus that underlies the phenotype is sufficient to show correlation. Similarly, a tissue or cell autonomous trait is correlated with an individual trait when the individual trait and the cell or tissue autonomous phenotype are found together with a higher than random statistical association. Thus, an hypothesis regarding a linkage between a cell or tissue phenotype and a trait is validated by performing an association study that shows a statistical linkage between the cell or tissue autonomous phenotype and the individual trait. This linkage can be detected by screening individuals for the cell or tissue autonomous trait, e.g., in a high-throughput cell based assay, or by detecting association between a genotype (that has been shown to be linked to the cell or tissue autonomous phenotype) and the individual trait.

Once initial correlations are established, the methods optionally include compiling and referencing a look up table that comprises correlation information between alleles of a polymorphism and the cellular/tissue response or phenotype and/or between the cellular/tissue response and a trait of an individual. The table can include data for multiple allele-phenotype and/or individual trait relationships and can take account of additive or other higher order effects of multiple allele-phenotype or response relationships, e.g., through the use of statistical tools such as principle component analysis, heuristic algorithms, etc.

Correlation of a marker to a cellular phenotype or individual trait optionally includes performing one or more statistical tests for correlation. Many statistical tests are known, and most are computer-implemented for ease of analysis. A variety of statistical methods of determining associations/correlations between traits and markers are known and can be applied to the present invention. For an introduction to the topic, see, Hartl (1981) A Primer of Population Genetics Washington University, Saint Louis Sinauer Associates, Inc. Sunderland, Mass. ISBN: 0-087893-271-2. A variety of appropriate statistical models are described in Lynch and Walsh (1998) Genetics and Analysis of Quantitative Traits, Sinauer Associates, Inc. Sunderland Mass. ISBN 0-87893-481-2. These models can, for example, be adapted to provide for correlations between genotype and cellular response values (rather than simply correlations between genotype and traits of an individual), characterize the influence of a locus on a cell or tissue response, sort out the relationship between environment and genotype, determine dominance or penetrance of genes, determine maternal and other epigenetic effects, determine principle components in an analysis (via principle component analysis, or “PCA”), and the like. The references cited in these texts provides considerable further detail on statistical models for correlating markers and phenotype.

In addition to standard statistical methods for determining correlation, other methods that determine correlations by pattern recognition and training, such as through the use of genetic algorithms, can be used to determine correlations between markers and cell or tissue responses. This is particularly useful when identifying higher order correlations between multiple alleles and responses (or multiple responses). To illustrate, neural network approaches can be coupled to genetic algorithm-type programming for heuristic development of a structure-function data space model that determines correlations between genetic information and phenotypic outcomes. For example, NNUGA (Neural Network Using Genetic Algorithms) is an available program (e.g., on the world wide web at cs.bgu.ac.il/˜omri/NNUGA) which couples neural networks and genetic algorithms. An introduction to neural networks can be found, e.g., in Kevin Gurney, An Introduction to Neural Networks, UCL Press (1999) and on the world wide web at shef.ac.uk/psychology/gurney/notes/index.html. Additional useful neural network references include those noted above in regard to genetic algorithms and, e.g., Bishop, Neural Networks for Pattern Recognition, Oxford University Press (1995), and Ripley et al., Pattern Recognition and Neural Networks, Cambridge University Press (1995).

Additional references that are useful in understanding data analysis applications for using and establishing correlations, principle components of an analysis, neural network modeling and the like, include, e.g., Hinchliffe, Modeling Molecular Structures, John Wiley and Sons (1996), Gibas and Jambeck, Bioinformatics Computer Skills, O'Reilly (2001), Pevzner, Computational Molecular Biology and Algorithmic Approach, The MIT Press (2000), Durbin et al., Biological Sequence Analysis: Probabilistic Models of Proteins and Nucleic Acids, Cambridge University Press (1998), and Rashidi and Buehler, Bioinformatic Basics: Applications in Biological Science and Medicine, CRC Press LLC (2000).

In any case, essentially any statistical test can be applied in a computer implemented model to establish correlations, by standard programming methods, or using any of a variety of “off the shelf” software packages that perform such statistical analyses, including, for example, those noted above and those that are commercially available, e.g., from Partek Incorporated (St. Peters, Mo.; www(dot)partek.com), e.g., that provide software for pattern recognition (e.g., which provide Partek Pro 2000 Pattern Recognition Software) which can be applied to genetic algorithms for multivariate data analysis, interactive visualization, variable selection, neural network & statistical modeling, etc. Relationships can be analyzed, e.g., by Principal Components Analysis (PCA) mapped mapped scatterplots and biplots, Multi-Dimensional Scaling (MDS) Multi-Dimensional Scaling (MDS) mapped scatterplots, star plots, etc. Available software for performing correlation analysis includes SAS, R and MathLab.

The marker(s) that are identified in the correlation studies, whether polymorphisms or cell responses, can be used for any of a variety of correlation analyses. For example, once polymorphic genetic markers have been identified, they can be used in a number of different assays for association studies, or as diagnostics, once an association has been validated. For example, probes can be designed for microarrays that interrogate these markers. Other exemplary assays include, e.g., the Taqman assays and molecular beacon assays, as well as conventional PCR and/or sequencing techniques.

Additional details regarding association studies can be found in U.S. Pat. No. 6,969,589, entitled “Methods for Genomic Analysis;” Ser. No. 10/042,819, filed Jan. 7, 2002, entitled “Genetic Analysis Systems and Methods;” Ser. No. 10/286,417, filed Oct. 31, 2002, entitled “Methods for Genomic Analysis;” Ser. No. 10/768,788, filed Jan. 30, 2004, entitled “Apparatus and Methods for Analyzing and Characterizing Nucleic Acid Sequences;” Ser. No. 10/447,685, filed May 28, 2003, entitled “Liver Related Disease Compositions and Methods;” Ser. No. 10/970,761, filed Oct. 20, 2004, entitled “Improved Analysis Methods and Apparatus for Individual Genotyping” (methods for individual genotyping); Ser. No. 10/956,224, filed Sep. 30, 2004, entitled “Methods for Genetic Analysis.”

In some embodiments, cell phenotype data is used to perform association studies to show correlations between genetic markers and cell or tissue phenotypes. This can be accomplished by determining genotypes of individuals with the cell or tissue response of interest (e.g., in cells or tissues corresponding to individuals or populations displaying the response of interest) and comparing the allele frequency or other characteristics (expression levels, etc.) of the genes of interest in these individuals to the allele frequency or other characteristics in a control group of cells. Such marker determinations can be conducted on a genome-wide basis, or can be focused on specific regions of the genome (e.g., haplotype blocks of interest).

In addition to the other embodiments of the methods of the present invention disclosed herein, the methods additionally allow for the “dissection” of a cell response. That is, particular cell responses can result from two or more different QTL. Scanning a plurality of QTL markers (e.g., as in genome or haplotype block scanning) allows for the dissection of varying genetic bases for similar (or graduated) cell responses.

One method of conducting association studies is to compare the allele frequency (or expression level) of genetic markers in cell lines or tissue cultures with a cellular phenotype of interest (“case group”) to the allele frequency in a control group of cell lines. In one method, informative SNPs (a.k.a. “tag SNPs”) are used to make the SNP haplotype pattern comparison (an “informative SNP” is a genetic SNP marker such as a SNP or subset (more than one) of SNPs in a genome or haplotype block that tends to distinguish one SNP or genome or haplotype pattern from other SNPs, genomes or haplotype patterns). The approach of using informative SNPs has an advantage over other whole genome scanning or genotyping methods, because, instead of reading all 3 billion bases of each cell line's genome, or even reading the 3-4 million common SNPs that may be found, only informative SNPs need to be detected (in human cell lines, a set of ˜250,000 informative SNPs have been identified (Hinds, et al. (2005) “Whole-Genome Patterns of Common DNA Variation in Three Human Populations,” Science 307:1072-1079)). Genotyping these particular, informative SNPs provides sufficient information to allow statistically accurate association data to be extracted from specific experimental cell populations, as described above.

Thus, in an embodiment of one method of determining genetic associations, the allele frequency for informative SNPs is determined for genomes of a control cell population that do not display the response, or that are “low responders” as described herein. The allele frequency of informative SNPs is also determined for genomes of a population that do display the phenotype, or that are “high responders.” The informative SNP allele frequencies are compared. Allele frequency comparisons can be made, for example, by determining the allele frequency (number of instances of a particular allele in a population divided by the total number of alleles) at each informative SNP location in each population and comparing these allele frequencies. The informative SNPs displaying a difference between the allele frequency of occurrence in the control versus case populations/groups are selected for analysis. Once informative SNPs are selected, the SNP haplotype block(s) or linkage disequilibrium bins (LD bins) that contain the informative SNPs are identified, which in turn identifies a genomic region of interest that is correlated with the phenotype. The genomic regions can be analyzed by genetic or any biological methods known in the art e.g., for use as drug discovery targets or as diagnostic markers.

Thus, the invention provides methods of correlating a cell or tissue autonomous phenotype to a genotype. The methods include detecting variance in a cell or tissue autonomous phenotype in a population of cells and/or tissues and accessing genotype information for the cells and/or tissues in the population. The variance is correlated to the genotype information, thereby correlating the cell or tissue autonomous phenotype and the genotype.

Typically, the cell or tissue autonomous phenotype is detected a plurality of times for each tissue or cell type, clone, or line within a population of cells or tissues, thereby amplifying correlation certainty during correlation events. For example, cell or tissue response can be detected about 100 or more times for each cell or tissue type in replicate experiments (e.g., using standard 96 or 384 well cell assay formats). This ability to detect cell and/or tissue autonomous response phenotypes multiple times for any correlation event is a significant advantage over standard correlation associations between traits and genotypes, where replicate studies for a given individual may be difficult, impractical, or impossible to perform.

Replicate analysis of cell and tissue phenotypes makes it possible to detect small variations in cell or tissue phenotypes in a population and to correlate them to genotypes. Thus, because correlations can be verified by replicate analysis, relatively small variations in cell or tissue phenotypes can be screened for and correlated to genotypes. This makes it possible to detect small additive effects of particular polymorphisms that are missed in standard genotype-trait correlation studies. Thus, for example, a portion of the polymorphisms detected optionally display 10% or less phenotypic variation in a population of cells.

Detecting the genotype optionally includes detecting a set of selected polymorphisms that correlate, positively or negatively, to a disease, a predisposition to the disease, a prognosis of the disease, and/or a treatment response or efficacy for the disease, e.g., based on the correlations established between genotype and cell phenotypes as described. In this context, a “favorable allele” is an allele at a particular locus that positively correlates with a desirable cellular or organismal phenotype, e.g., depending on context, resistance to radiation damage, or that negatively correlates with an undesirable cellular or organismal phenotype, e.g., depending on context, susceptibility to radiation damage. The desired phenotype can, of course, vary, e.g., in different contexts a drug response can be desirable or undesirable. A favorable allele of a linked marker is a marker that segregates with the favorable allele. A favorable allelic form of a chromosome segment is a chromosome segment that includes a nucleotide sequence that positively correlates with the desired phenotype, or that negatively correlates with the unfavorable phenotype at one or more genetic loci physically located on the chromosome segment.

Once a cell or tissue phenotype is correlated to a genotype, the genotype can be used as a marker for the phenotype, e.g., to validate an hypothesis regarding a correlation between the cell or tissue phenotype and an individual trait. That is, a population that displays variance in the trait of interest is examined to detect correlations in the population between the marker and the individual trait, e.g., using standard statistical methods for examining association. While detection of a genotype marker is generally simpler than directly detecting the cell or tissue autonomous trait, it is also possible in this case to directly examine a correlation between an individual trait and the cell or tissue autonomous phenotype. In this approach, a cell or tissue based assay is used to screen individuals of the population for a cell or tissue phenotype. In this embodiment, a high-throughput assay system that can monitor the phenotype is desirably employed. Associations between the cell or tissue phenotype and the individual trait of interest are determined by standard statistical methods.

Results from a cell or tissue based study can also be used in multiple validation studies for different organismal phenotypes. That is, the same cellular phenotype can underlie multiple different organismal phenotypes. For example, if a cell displays a slow response to DNA damage as measured in a DNA damage repair assay, an individual may display increased susceptibility to many different cancers. Similarly, an abnormal insulin response in cells of an individual may be prognostic for susceptibility to diabetes, metabolic syndrome, obesity, decreased lifespan, circulatory problems, increased risk of heart disease, etc.

Whether the association study is performed to identify correlations between a genotype and a cell or tissue autonomous phenotype, or between a trait and the genotype or phenotype, a database of correlation relationships can be assembled. For example, a database of correlations (e.g., correlations determined by the methods presented herein) can include a lookup table that comprises correlation relationships for the cell autonomous phenotype and/or genotype and the trait, e.g., disease, predisposition to the disease, prognosis of the disease, and/or the treatment response or efficacy for the disease, etc. The database is optionally a heuristic database that refines correlations between genotype, cell autonomous phenotype, and/or the trait, e.g., disease, predisposition to the disease, etc., based upon inputs regarding the correlation. The heuristic database can include a neural network (NN), a statistical model (SM), a hidden Markov model (HMM), principal component analysis (PCA), classification and regression trees (CART), multivariate adaptive regression splines (MARS), genetic algorithms (GA), multiple linear regression (MLR), variable importance for projection (VIP), inverse least squares (ILS), partial least square (PLS) or any other suitable process or statistical framework, e.g., as noted in the references above.

In general, correlations between genotype, cell or tissue autonomous phenotype, and/or a patient condition can be determined (or predicted) by the methods herein. This allows for the identification of correlations between any of a variety of diseases or treatment outcomes and a genotype or cellular phenotype (e.g., correlation to a treatment side effect, a side effect predisposition, disease state, a disease predisposition, a disease prognosis, a disease treatment response a disease treatment efficacy, etc.). This information can be used for diagnosing, detecting, detecting a predisposition for, predicting an outcome of and/or selecting a treatment regimen for a disease in a patient base upon a correlation between a cell autonomous phenotype of the patient and the genotype.

### Applications

The methods of the invention optionally can include validating an hypothesis regarding an association between the cell or tissue autonomous trait (or marker thereof) and a trait of interest. Once this validation is performed, the correlation information serves as a diagnostic for the trait. Thus, the invention also includes methods for diagnosing, detecting, detecting a predisposition for, predicting an outcome of and/or selecting a treatment regimen for a disease (e.g., taking cell-autonomous drug effects into account). These methods include, e.g., detecting at least one in vitro cell or tissue autonomous phenotype of a cell or tissue from a patient and/or detecting a genotype correlated to the cell or tissue autonomous phenotype, and accessing a database comprising correlation information regarding a correlation between the cell or tissue autonomous phenotype and/or genotype and, e.g., one or more of: disease, predisposition to the disease, prognosis of the disease, and/or treatment efficacy or response for or to the disease. Based on the correlation, the method includes diagnosing, detecting, detecting a predisposition for, predicting an outcome of and/or selecting a treatment regimen for the disease.

Methods of determining genotype information for a cell line, a tissue, or a patient are known, and include any available method for detecting polymorphisms in the patient. These include, e.g., hybridization of nucleic acids derived from the patient to an array designed to detect polymorphisms, and/or amplification of nucleic acids of the patient, e.g., real-time amplification, to detect polymorphisms in amplified nucleic acids.

In one aspect, only one or a few correlated (“associated”) polymorphisms need to be screened in additional individuals to determine if they will exhibit the organismal phenotype. This makes it possible to provide a simple and convenient diagnostic application for detecting the organismal phenotype of interest. For example, the set of selected polymorphisms optionally includes fewer than 100 polymorphisms, or even less than 10 polymorphisms, greatly simplifying detection formats. Further details regarding appropriate diagnostic and other application formats are found, e.g., below.

Marker Amplification Strategies

In some embodiments, markers for one or a few polymorphisms can provide sufficient information to determine if the patient is likely to exhibit or develop the organismal phenotype of interest. Thus, rather than rescreening large numbers of polymorphisms for each patient, one or a few polymorphisms are interrogated, to provide a “bar code” of the relevant genotype for the patient (See, e.g., PCT patent application US05/007375, filed Mar. 3, 2005, entitled “Methods for Genetic Analysis”). This is conveniently done using amplification technologies, e.g., detecting the relevant polymorphisms in amplicons derived from the patient.

Amplification primers for amplifying such informative markers (e.g., informative marker loci) and suitable probes to detect such markers or to genotype a sample with respect to multiple marker alleles, are, thus, a feature of the invention. Once relevant polymorphisms are defined in the correlation analysis described above (identifying polymorphisms that correlate to the cell autonomous phenotype of interest), one of skill can design amplification primers that amplify the polymorphic sequence or a portion thereof. For example, primer selection for long-range PCR is described in U.S. Ser. No. 10/042,406, filed Jan. 9, 2002 and U.S. Ser. No. 10/236,480, filed Sep. 5, 2002; for short-range PCR, U.S. Ser. No. 10/341,832, filed Jan. 14, 2003 provides guidance with respect to primer selection. Also, there are publicly available programs such as “Oligo” available for primer design. Thus, for example for the identification of human polymorphisms, with such available primer selection and design software, the publicly available human genome sequence and the polymorphism locations as provided by the correlation analysis, one of skill can design primers to amplify, e.g., SNPs that correlate to a cell or tissue autonomous phenotype.

It will also be appreciated that the precise primers and/or probes used for detection of a nucleic acid comprising a SNP or other polymorphism (e.g., an amplicon comprising the polymorphism) can vary, e.g., any primer or probe that can produce and/or identify the polymorphic region of a marker amplicon to be detected can be used in conjunction with the present invention. Further, the configuration of amplification primers and detection probes can, of course, vary. Thus, the invention is not limited to particular sequences.

Indeed, it will be appreciated that amplification is not a requirement for marker detection. For example, one can directly detect unamplified genomic DNA simply by performing a Southern blot on a sample of genomic DNA. Procedures for performing Southern blotting, standard amplification (PCR, LCR, or the like) and many other nucleic acid detection methods are well established and are taught, e.g., in Sambrook et al., Molecular Cloning—A Laboratory Manual (3rd Ed.), Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)) and PCR Protocols A Guide to Methods and Applications (Innis et al. eds) Academic Press Inc. San Diego, Calif. (1990) (Innis).

Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization, discrimination on a sizing gel, or the like).

Typically, molecular markers are detected by any established method available in the art, including, without limitation, allele specific hybridization (ASH), detection of single nucleotide extensions, array hybridization (optionally including ASH), or other methods for detecting single nucleotide polymorphisms (SNPs), amplified fragment length polymorphism (AFLP) detection, amplified variable sequence detection, randomly amplified polymorphic DNA (RAPD) detection, restriction fragment length polymorphism (RFLP) detection, self-sustained sequence replication detection, simple sequence repeat (SSR) detection, single-strand conformation polymorphisms (SSCP) detection, isozyme marker detection, northern analysis (e.g., where expression levels are used as markers), quantitative amplification of mRNA or cDNA, or the like. While SNP markers provide one preferred class of marker, any of the aforementioned marker types can be employed in the context of the invention to identify linked loci that affect or effect the cell or tissue autonomous phenotype.

Example Techniques For Detection of Polymorphisms

The invention provides methods of identifying molecular markers that comprise or are linked to QTL for cell or tissue-autonomous phenotypes. The markers find use in diagnosing disease, predisposition to disease, prognosis of disease, and/or treatment efficacy or response for or to the disease, as well as for marker assisted selection for desired traits in crops and livestock. It is not intended that the invention be limited to any particular method for the detection of these markers.

Markers corresponding to genetic polymorphisms between members of a population can be detected by numerous methods well-established in the art (e.g., PCR-based sequence specific amplification, restriction fragment length polymorphisms (RFLPs), isozyme markers, northern analysis, allele specific hybridization (ASH), array based hybridization, amplified variable sequences of the genome, self-sustained sequence replication, simple sequence repeat (SSR), single nucleotide polymorphism (SNP), random amplified polymorphic DNA (“RAPD”) or amplified fragment length polymorphisms (AFLP). In one additional embodiment, the presence or absence of a molecular marker is determined simply through nucleotide sequencing of the polymorphic marker region. Any of these methods are readily adapted to high throughput analysis.

Some techniques for detecting genetic markers utilize hybridization of a probe nucleic acid to nucleic acids corresponding to the genetic marker (e.g., amplified nucleic acids produced using genomic DNA as a template). Hybridization formats, including, but not limited to: solution phase, solid phase, mixed phase, or in situ hybridization assays are useful for allele detection. An extensive guide to the hybridization of nucleic acids is found in Tijssen (1993) Laboratory Techniques in Biochemistry and Molecular Biology—Hybridization with Nucleic Acid Probes Elsevier, New York, as well as in Sambrook, Berger and Ausubel.

For example, markers that comprise restriction fragment length polymorphisms (RFLP) are detected, e.g., by hybridizing a probe which is typically a sub-fragment (or a synthetic oligonucleotide corresponding to a sub-fragment) of the nucleic acid to be detected to restriction digested genomic DNA. The restriction enzyme is selected to provide restriction fragments of at least two alternative (or polymorphic) lengths in different individuals or populations. Determining one or more restriction enzyme that produces informative fragments for each allele of a marker is a simple procedure, well known in the art. After separation by length in an appropriate matrix (e.g., agarose or polyacrylamide) and transfer to a membrane (e.g., nitrocellulose, nylon, etc.), the labeled probe is hybridized under conditions which result in equilibrium binding of the probe to the target followed by removal of excess probe by washing.

Nucleic acid probes to the marker loci can be cloned and/or synthesized. Any suitable label can be used with a probe of the invention. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radiolabels, enzymes, and colorimetric labels. Other labels include ligands which bind to antibodies labeled with fluorophores, chemiluminescent agents, and enzymes. A probe can also constitute radiolabelled PCR primers that are used to generate a radiolabelled amplicon. Labeling strategies for labeling nucleic acids and corresponding detection strategies can be found, e.g., in Haugland (2003) Handbook of Fluorescent Probes and Research Chemicals Ninth Edition by Molecular Probes, Inc. (Eugene Oreg.). Additional details regarding marker detection strategies are found below.

Amplification-Based Detection Methods

PCR, RT-PCR and LCR are in particularly broad use as amplification and amplification-detection methods for amplifying nucleic acids of interest (e.g., those comprising marker loci), facilitating detection of the nucleic acids of interest. Details regarding the use of these and other amplification methods can be found in any of a variety of standard texts, including, e.g., Sambrook, Ausubel, and Berger. Many available biology texts also have extended discussions regarding PCR and related amplification methods. One of skill will appreciate that essentially any RNA can be converted into a double stranded DNA suitable for restriction digestion, PCR expansion and sequencing using reverse transcriptase and a polymerase (“Reverse Transcription-PCR, or “RT-PCR”). See also, Ausubel, Sambrook and Berger, above. These methods can also be used to quantitatively amplify mRNA or corresponding cDNA, providing an indication of expression levels of mRNA that correspond to any polymorphism of interest. Differences in expression levels for these genes between individuals, families, lines and/or populations can be used as markers for a cell or tissue autonomous phenotype (or can themselves comprise the cell or tissue autonomous phenotype, e.g., where the phenotype is simply an expression level of a particular gene).

Real Time Amplification/Detection Methods

In one aspect, real time PCR or LCR is performed on the amplification mixtures described herein, e.g., using molecular beacons or TaqMan™ probes. A molecular beacon (MB) is an oligonucleotide or PNA which, under appropriate hybridization conditions, self-hybridizes to form a stem and loop structure. The MB has a label and a quencher at the termini of the oligonucleotide or PNA; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, e.g., to a region of an amplicon during amplification), the MB label is unquenched. Details regarding standard methods of making and using MBs are well established in the literature and MBs are available from a number of commercial reagent sources. See also, e.g., Leone et al. (1995) “Molecular beacon probes combined with amplification by NASBA enable homogenous real-time detection of RNA.” Nucleic Acids Res. 26:2150-2155; Tyagi and Kramer (1996) “Molecular beacons: probes that fluoresce upon hybridization” Nature Biotechnology 14:303-308; Blok and Kramer (1997) “Amplifiable hybridization probes containing a molecular switch” Mol Cell Probes 11:187-194; Hsuih et al. (1997) “Novel, ligation-dependent PCR assay for detection of hepatitis C in serum” J Clin Microbiol 34:501-507; Kostrikis et al. (1998) “Molecular beacons: spectral genotyping of human alleles” Science 279:1228-1229; Sokol et al. (1998) “Real time detection of DNA:RNA hybridization in living cells” Proc. Natl. Acad. Sci. U.S.A. 95:11538-11543; Tyagi et al. (1998) “Multicolor molecular beacons for allele discrimination” Nature Biotechnology 16:49-53; Bonnet et al. (1999) “Thermodynamic basis of the chemical specificity of structured DNA probes” Proc. Natl. Acad. Sci. U.S.A. 96:6171-6176; Fang et al. (1999) “Designing a novel molecular beacon for surface-immobilized DNA hybridization studies” J. Am. Chem. Soc. 121:2921-2922; Marras et al. (1999) “Multiplex detection of single-nucleotide variation using molecular beacons” Genet. Anal. Biomol. Eng. 14:151-156; and Vet et al. (1999) “Multiplex detection of four pathogenic retroviruses using molecular beacons” Proc. Natl. Acad. Sci. U.S.A. 96:6394-6399. Additional details regarding MB construction and use is found in the patent literature, e.g., U.S. Pat. No. 5,925,517 (Jul. 20, 1999) to Tyagi et al. entitled “Detectably labeled dual conformation oligonucleotide probes, assays and kits;” U.S. Pat. No. 6,150,097 to Tyagi et al (Nov. 21, 2000) entitled “Nucleic acid detection probes having non-FRET fluorescence quenching and kits and assays including such probes” and U.S. Pat. No. 6,037,130 to Tyagi et al (Mar. 14, 2000), entitled “Wavelength-shifting probes and primers and their use in assays and kits.”

PCR detection and quantification using dual-labeled fluorogenic oligonucleotide probes, commonly referred to as “TaqMan™” probes, can also be performed according to the present invention. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. Accordingly, TaqMan™ probes are oligonucleotides that have a label and a quencher, where the label is released during amplification by the exonuclease action of the polymerase used in amplification. This provides a real time measure of amplification during synthesis. A variety of TaqMan™ reagents are commercially available, e.g., from Applied Biosystems (Division Headquarters in Foster City, Calif.) as well as from a variety of specialty vendors such as Biosearch Technologies (e.g., black hole quencher probes). Further details regarding dual-label probe strategies can be found, e.g., in WO92/02638.

Other similar methods include e.g. fluorescence resonance energy transfer between two adjacently hybridized probes, e.g., using the “LightCycler®” format described in U.S. Pat. No. 6,174,670.

Array-Based Marker Detection

As noted, array-based detection of polymorphisms for diagnostic and other applications can be performed using commercially available arrays, e.g., from Affymetrix (Santa Clara, Calif.) or other manufacturers. Further details regarding array-based detection are found above, e.g., in the context of determining genotype information for correlation analysis; the previous discussion is equally applicable to diagnostic and other applications that monitor genotype information.

Additional Details Regarding Amplified Variable Sequences, SSR, AFLP ASH, SNPs and Isozyme Markers

Amplified variable sequences refer to amplified sequences of the genome that exhibit high nucleic acid residue variability between members of the same species. All organisms have variable genomic sequences and each organism (with the exception of a clone) has a different set of variable sequences. Once identified, the presence of specific variable sequence can be used to predict phenotypic traits, including cell-autonomous and tissue autonomous traits. Preferably, DNA from the genome serves as a template for amplification with primers that flank a variable sequence of DNA. The variable sequence is amplified and then sequenced.

Alternatively, self-sustained sequence replication can be used to identify genetic markers. Self-sustained sequence replication refers to a method of nucleic acid amplification using target nucleic acid sequences which are replicated exponentially, in vitro, under substantially isothermal conditions by using three enzymatic activities involved in retroviral replication: (1) reverse transcriptase, (2) Rnase H, and (3) a DNA-dependent RNA polymerase (Guatelli et al. (1990) Proc Natl Acad Sci USA 87:1874). By mimicking the retroviral strategy of RNA replication by means of cDNA intermediates, this reaction accumulates cDNA and RNA copies of the original target.

Amplified fragment length polymophisms (AFLP) can also be used as genetic markers (Vos et al. (1995) Nucl Acids Res 23:4407). The phrase “amplified fragment length polymorphism” refers to selected restriction fragments which are amplified before or after cleavage by a restriction endonuclease. The amplification step allows easier detection of specific restriction fragments. AFLP allows the detection large numbers of polymorphic markers and has been used for genetic mapping (Becker et al. (1995) Mol Gen Genet 249:65; and Meksem et al. (1995) Mol Gen Genet 249:74).

Allele-specific hybridization (ASH) can be used to identify the genetic markers of the invention. ASH technology is based on the stable annealing of a short, single-stranded, oligonucleotide probe to a completely complementary single-strand target nucleic acid. Detection may be accomplished via an isotopic or non-isotopic label attached to the probe.

For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization. In this manner, only one of the alternative probes will hybridize to a target sample that is homozygous or homogenous for an allele. Samples that are heterozygous or heterogeneous for two alleles will hybridize to both of two alternative probes.

ASH markers are used as dominant markers where the presence or absence of only one allele is determined from hybridization or lack of hybridization by only one probe. The alternative allele may be inferred from the lack of hybridization. ASH probe and target molecules are optionally RNA or DNA; the target molecules are any length of nucleotides beyond the sequence that is complementary to the probe; the probe is designed to hybridize with either strand of a DNA target; the probe ranges in size to conform to variously stringent hybridization conditions, etc.

PCR allows the target sequence for ASH to be amplified from low concentrations of nucleic acid in relatively small volumes. Otherwise, the target sequence from genomic DNA is digested with a restriction endonuclease and size separated by gel electrophoresis. Hybridizations typically occur with the target sequence bound to the surface of a membrane or, as described in U.S. Pat. No. 5,468,613, the ASH probe sequence may be bound to a membrane.

In one embodiment, ASH data are typically obtained by amplifying nucleic acid fragments (amplicons) from genomic DNA using PCR, transferring the amplicon target DNA to a membrane in a dot-blot format, hybridizing a labeled oligonucleotide probe to the amplicon target, and observing the hybridization dots by autoradiography.

Single nucleotide polymorphisms (SNP) are markers that consist of a shared sequence differentiated on the basis of a single nucleotide. Typically, this distinction is detected by differential migration patterns of an amplicon comprising the SNP on e.g., an acrylamide gel. However, alternative modes of detection, such as hybridization, e.g., ASH, or RFLP analysis are also appropriate.

Isozyme markers can be employed as genetic markers, e.g., to track isozyme markers linked to the markers herein. Isozymes are multiple forms of enzymes that differ from one another in their amino acid, and therefore their nucleic acid sequences. Some isozymes are multimeric enzymes contain slightly different subunits. Other isozymes are either multimeric or monomeric but have been cleaved from the proenzyme at different sites in the amino acid sequence. Isozymes can be characterized and analyzed at the protein level, or alternatively, isozymes which differ at the nucleic acid level can be determined. In such cases any of the nucleic acid based methods described herein can be used to analyze isozyme markers.

Additional Details Regarding Nucleic Acid Amplification

As noted, nucleic acid amplification techniques such as PCR and LCR are well known in the art and can be applied to the present invention to amplify and/or detect nucleic acids of interest, such as nucleic acids comprising marker loci. Examples of techniques sufficient to direct persons of skill through such in vitro methods, including the polymerase chain reaction (PCR), the ligase chain reaction (LCR), Qβ-replicase amplification and other RNA polymerase mediated techniques (e.g., NASBA), are found in the references noted above, e.g., Innis, Sambrook, Ausubel, and Berger. Additional details are found in Mullis et al. (1987) U.S. Pat. No. 4,683,202; Amheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of amplifying large nucleic acids by PCR, which is useful in the context of positional cloning, are further summarized in Cheng et al. (1994) Nature 369: 684, and the references therein, in which PCR amplicons of up to 40 kb are generated. Methods for long-range PCR are disclosed, for example, in U.S. patent application Ser. No. 10/042,406, filed Jan. 9, 2002, entitled “Algorithms for Selection of Primer Pairs”; U.S. patent application Ser. No. 10/236,480, filed Sep. 9, 2002, entitled “Methods for Amplification of Nucleic Acids”; and U.S. Pat. No. 6,740,510, issued May 25, 2004, entitled “Methods for Amplification of Nucleic Acids”. U.S. Ser. No. 10/341,832 (filed Jan. 14, 2003) also provides details regarding primer picking methods for performing short range PCR.

Detection of Protein Expression Products

Proteins that correspond to marker nucleic acids can be correlated to the phenotypes of interest herein. For a description of the basic paradigm of molecular biology, including the expression (transcription and/or translation) of DNA into RNA into protein, see, Alberts et al. (2002) Molecular Biology of the Cell, 4^{th} Edition Taylor and Francis, Inc., ISBN: 0815332181 (“Alberts”), and Lodish et al. (1999) Molecular Cell Biology, 4^{th} Edition W H Freeman & Co, ISBN: 071673706X (“Lodish”). Accordingly, proteins corresponding to any marker linked to a cell or tissue autonomous trait (or that constitute the trait, where the trait is a particular expression product of a cell or tissue) can be detected as markers, e.g., by detecting different protein isotypes between individuals or populations, or by detecting a differential presence, absence or expression level of such a protein of interest.

A variety of protein detection methods are known and can be used to distinguish protein markers. In addition to the various references noted supra, a variety of protein manipulation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000).

“Proteomic” detection methods, which detect many proteins simultaneously have been described. These can include various multidimensional electrophoresis methods (e.g., 2-d gel electrophoresis), mass spectrometry based methods (e.g., SELDI, MALDI, electrospray, etc.), or surface plasmon reasonance methods. For example, in MALDI, a sample is usually mixed with an appropriate matrix, placed on the surface of a probe and examined by laser desorption/ionization. The technique of MALDI is well known in the art. See, e.g., U.S. Pat. No. 5,045,694 (Beavis et al.), U.S. Pat. No. 5,202,561 (Gleissmann et al.), and U.S. Pat. No. 6,111,251 (Hillenkamp). Similarly, for SELDI, a first aliquot is contacted with a solid support-bound (e.g., substrate-bound) adsorbent. A substrate is typically a probe (e.g., a biochip) that can be positioned in an interrogatable relationship with a gas phase ion spectrometer. SELDI is also a well known technique, and has been applied to diagnostic proteomics. See, e.g. Issaq et al. (2003) “SELDI-TOF MS for Diagnostic Proteomics” Analytical Chemistry 75: 149A-155A.

In general, the above methods can be used to detect different forms (alleles) of proteins and/or can be used to detect different expression levels of the proteins (which can be due to allelic differences) between cells, tissues, individuals, families, lines, populations, etc. Differences in expression levels, when controlled for environmental factors, can be indicative of different alleles at a QTL for the gene of interest, even if the encoded differentially expressed proteins are themselves identical. This occurs, for example, where there are multiple allelic forms of a gene in non-coding regions, e.g., regions such as promoters or enhancers that control gene expression. Thus, detection of differential expression levels can be used as a method of detecting allelic differences.

In other aspect of the present invention, a gene comprising, in linkage disequilibrium with, or under the control of a nucleic acid associated with a cell or tissue autonomous phenotype may exhibit differential allelic expression. “Differential allelic expression” as used herein refers to both qualitative and quantitative differences in the allelic expression of multiple alleles of a single gene present in a cell. As such, a gene displaying differential allelic expression may have one allele expressed at a different time or level as compared to a second allele in the same cell/tissue. Differential allelic expression and analysis methods are disclosed in detail in U.S. patent application Ser. No. 10/438,184, filed May 13, 2003 and U.S. patent application Ser. No. 10/845,316, filed May 12, 2004, both of which are entitled “Allele-specific expression patterns.” Detection of a differential allelic expression pattern of one or more nucleic acids, or fragments, derivatives, polymorphisms, variants or complements thereof, associated with a cell or tissue autonomous phenotype is diagnostic for any individual trait associated with the cell or tissue autonomous phenotype.

Additional Details Regarding Types of Markers Appropriate for Screening

Biological markers that are screened for correlation to the phenotypes herein can be any of those types of markers that can be detected by screening, e.g., genetic markers such as allelic variants of a genetic locus (e.g., as in SNPs), expression markers (e.g., presence or quantity of mRNAs and/or proteins), and/or the like.

The nucleic acid of interest to be amplified, transcribed, translated and/or detected in the methods of the invention can be essentially any nucleic acid, though nucleic acids derived from human sources are especially relevant to the detection of markers associated with disease diagnosis and clinical applications for human patients. The sequences for many nucleic acids and amino acids (from which nucleic acid sequences can be derived via reverse translation) are available. Common sequence repositories for known nucleic acids include GenBank® EMBL, DDBJ and the NCBI. Other repositories can easily be identified by searching the internet. The nucleic acid to be amplified, transcribed, translated and/or detected can be an RNA (e.g., where amplification includes RT-PCR or LCR, the Van-Gelder Eberwine reaction or Ribo-SPIA) or DNA (e.g., amplified DNA, cDNA or genomic DNA), or even any analogue thereof (e.g., for detection of synthetic nucleic acids or analogues thereof, e.g., where the sample of interest includes or is used to derive or synthesize artificial nucleic acids). Any variation in a nucleic acid sequence or expression level between individuals or populations can be detected as a marker, e.g., a mutation, a polymorphism, a single nucleotide polymorphism (SNP), an allele, an isotype, expression of an RNA or protein, etc. One can detect variation in sequence, expression levels or gene copy numbers as markers that can be correlated to a cell autonomous phenotype of interest.

For example, the methods of the invention are useful in screening samples derived from patients for a marker nucleic acid of interest, e.g., from bodily fluids (blood, saliva, urine etc.), tissue, and/or waste from the patient. Thus, stool, sputum, saliva, blood, lymph, tears, sweat, urine, vaginal secretions, ejaculatory fluid or the like can easily be screened for nucleic acids by the methods of the invention, as can essentially any tissue of interest that contains the appropriate nucleic acids. These samples are typically taken, following informed consent, from a patient by standard medical laboratory methods.

Prior to amplification and/or detection of a nucleic acid comprising a marker, the nucleic acid is optionally purified from the samples by any available method, e.g., those taught in Berger and Kimmel, Sambrook and/or Ausubel. A plethora of kits are also commercially available for the purification of nucleic acids from cells or other samples (see, e.g., EasyPrep™, FlexiPrep™, both from Pharmacia Biotech; StrataClean™, from Stratagene; and, QIAprep™ from Qiagen). Alternately, samples can simply be directly subjected to amplification or detection, e.g., following aliquotting and/or dilution.

Examples of markers can include polymorphisms, single nucleotide polymorphisms, presence of one or more nucleic acids in a sample, absence of one or more nucleic acids in a sample, presence of one or more genomic DNA sequences, absence or one or more genomic DNA sequences, presence of one or more mRNAs, absence of one or more mRNAs, expression levels of one or more mRNAs, presence of one or more proteins, expression levels of one or more proteins, and/or data derived from any of the preceding or combinations thereof. Essentially any number of markers can be detected, using available methods, e.g., using array technologies that provide high density, high throughput marker mapping. Thus, at least about 10, 100, 1,000, 10,000, or even 100,000 or more genetic markers can be tested, simultaneously or in a serial fashion (or combination thereof), to detect a correlated phenotype. Combinations of markers can also be desirably tested, e.g., to identify genetic combinations or combinations of expression patterns in populations that are correlated to the phenotype.

As noted, the biological marker to be detected can be any detectable biological component. Commonly detected markers include genetic markers (e.g., DNA sequence markers present in genomic DNA or expression products thereof) and expression markers (which can reflect genetically coded factors, environmental factors, or both). Where the markers are expression markers, the methods can include determining a first expression profile for a first cell, tissue or individual or population (e.g., of one or more expressed markers, e.g., a set of expressed markers) and comparing the first expression profile to a second expression profile for a second cell, tissue, individual or population (e.g., comparing a case and a control). In this example, correlating expression marker(s) to a particular phenotype can include correlating the first or second expression profile to the phenotype of interest (whether the phenotype comprises a cell, tissue or individual trait).

Probe/Primer Synthesis Methods

In general, synthetic methods for making markers, e.g., oligonucleotides, including probes, primers, molecular beacons, PNAs, LNAs (locked nucleic acids), etc., are well known. For example, oligonucleotides can be synthesized chemically according to the solid phase phosphoramidite triester method described by Beaucage and Caruthers (1981), Tetrahedron Letts., 22(20):1859-1862, e.g., using a commercially available automated synthesizer, e.g., as described in Needham-VanDevanter et al. (1984) Nucleic Acids Res., 12:6159-6168. Oligonucleotides, including modified oligonucleotides can also be ordered from a variety of commercial sources known to persons of skill. There are many commercial providers of oligo synthesis services, and thus this is a broadly accessible technology. Any nucleic acid can be custom ordered from any of a variety of commercial sources, such as The Midland Certified Reagent Company (mcrc@oligos.com), The Great American Gene Company (www(dot)genco(dot)com), ExpressGen Inc. (www(dot)expressgen(dot)com), Operon Technologies Inc. (Alameda, Calif.) and many others. Similarly, PNAs can be custom ordered from any of a variety of sources, such as PeptidoGenic (pkim@ccnet(dot)com), HTI Bio-products, inc. (htibio(dot)com), BMA Biomedicals Ltd (U.K.), Bio-Synthesis, Inc., and many others.

In Silico Marker Detection

In some embodiments, in silico methods can be used to detect the marker loci of interest. For example, the sequence of a nucleic acid comprising the marker locus of interest can be stored in a computer. The desired marker locus sequence or its homolog can be identified using an appropriate nucleic acid search algorithm as provided by, for example, in such readily available programs as BLAST, or even simple word processors. The entire human genome has been sequenced and, thus, sequence information can be used to identify marker regions, flanking nucleic acids, etc.

Amplification Primers For Marker Detection

In some preferred embodiments, the molecular markers of the invention are detected in diagnostic applications using a suitable PCR-based detection method, where the size or sequence of the PCR amplicon is indicative of the absence or presence of the marker (e.g., a particular marker allele). In these types of methods, PCR primers are hybridized to the conserved regions flanking the polymorphic marker region.

Suitable primers to be used with the invention can be designed using any suitable method. It is not intended that the invention be limited to any particular primer or primer pair. For example, primers can be designed using any suitable software program, such as LASERGENE®, e.g., taking account of publicly available sequence information.

In some embodiments, the primers of the invention are radiolabelled, or labeled by any suitable means (e.g., using a non-radioactive fluorescent tag), to allow for rapid visualization of the different size amplicons following an amplification reaction without any additional labeling step or visualization step. In some embodiments, the primers are not labeled, and the amplicons are visualized following their size resolution, e.g., following agarose or acrylamide gel electrophoresis. In some embodiments, ethidium bromide staining of the PCR amplicons following size resolution allows visualization of the different size amplicons.

Detection of Markers For Positional Cloning

In some embodiments, a nucleic acid probe is used to detect a nucleic acid that comprises a marker sequence, e.g., corresponding to a cell or tissue autonomous phenotype. Such probes can be used, for example, in positional cloning to isolate nucleotide sequences linked to the marker nucleotide sequence. It is not intended that the nucleic acid probes of the invention be limited to any particular size. In some embodiments, nucleic acid probe is at least 20 nucleotides in length, or alternatively, at least 50 nucleotides in length, or alternatively, at least 100 nucleotides in length, or alternatively, at least 200 nucleotides in length.

A hybridized probe is detected using, autoradiography, fluorography or other similar detection techniques depending on the label to be detected. Examples of specific hybridization protocols are widely available in the art, see, e.g., Berger, Sambrook, and Ausubel, all herein.

Further Details Regarding Diagnostic Applications

As discussed above, the cell autonomous phenotype can be an indicator for the physiological status of a patient. Such cell autonomous phenotypes include: a radiation response of the cell, a response of the cell to an anti-cancer drug, a response of the cell to a therapeutic agent, a measure of DNA damage repair by the cell, an immune response by the cell, an anti-inflammatory response of the cell, cytokine production by the cell, energy metabolism of the cell, oxygen consumption of the cell, an electrical response of the cell, a flow of ions across a membrane of the cell, apoptosis of the cell, an expression level of a housekeeping gene of the cell, an activity of a hosekeeping gene product, and cell cycle regulation of the cell. The relevant disease can include, e.g., cancer, an infectious disease, a viral infection, a bacterial infection, an immune disorder, an autoimmune disorder, obesity, diabetes, cardiovascular disease, a metabolic disorder, metabolic syndrome, a neurodegnerative disease, a CNS disorder, a transplant-related condition, and/or a genetic disease.

The treatment regimen for such a disease that is under consideration can be, e.g., surgery, exposure to radiation, administration of a drug, administration of an anti-cancer drug, administration of an anti-viral drug, administration of an antibiotic, administration of an immune suppressor or enhancer, administration of a cardiovascular drug, administration of a cholesterol level regulating drug, administration of a neurological drug, administration of an anti-rejection drug, administration of an enzyme inhibitor, administration of an enzyme activator, diet, and/or exercise.

Other Applications

As discussed above, clinical applications such as diagnostic detection of genotypes that correlate to cell or tissue autonomous phenotypes and/or organismal phenotypes provide a significant class of embodiments of the invention. Other significant embodiments include agricultural applications, such as marker assisted selection (MAS) of crops and/or livestock. For example, traits such as yield, oil content, sugar content, time to maturity, disease resistance or tolerance, pest resistance or tolerance, heat and cold tolerance, fat production, lean meat production, milk production, egg production, coat density, and many others are routinely selected for in the agricultural industry. To the extent that any such trait is linked to a cell or tissue-autonomous phenotype or genotype by the methods herein, MAS can be used to select for such traits as part of controlled breeding programs.

The methods of correlation and detection of linked markers noted throughout apply equally to this application. Once correlations are established, MAS is performed by standard MAS methods.

### Systems for Correlating Genotype and Cell Autonomous Phenotype

Systems for performing the above correlations are also a feature of the invention. For example, the system can include system instructions (e.g., computer implemented) that correlate the presence or absence of an allele with a cell or tissue autonomous phenotype. Optionally, the system instructions can compare detected information as to allele sequences or expression levels with a database that includes correlations between the alleles and the relevant phenotypes. As noted above, this database can be multidimensional, thereby including higher-order relationships between combinations of alleles and the relevant phenotypes. These relationships can be stored in any number of look-up tables, e.g., taking the form of spreadsheets (e.g., Excel™ spreadsheets) or databases such as an Access™, SQL™, Oracle™, Paradox™, or similar database. The system includes provisions for inputting sample-specific information regarding allele detection information, e.g., through an automated or user interface and for comparing that information to the look up tables.

Optionally, the system instructions can also include software that accepts diagnostic information associated with any detected allele information, e.g., a diagnosis that a subject with the relevant allele has a particular phenotype. This software can be heuristic in nature, using such inputted associations to improve the accuracy of the look up tables and/or interpretation of the look up tables by the system. A variety of such approaches, including neural networks, Markov modeling, and other statistical analysis are described above.

The invention provides data acquisition modules for detecting one or more detectable genetic marker(s) (e.g., one or more array comprising one or more biomolecular probes, detectors, fluid handlers, or the like). The biomolecular probes of such a data acquisition module can include any that are appropriate for detecting the biological marker, e.g., oligonucleotide probes, proteins, aptamers, antibodies, etc. These can include sample handlers (e.g., fluid handlers), robotics, microfluidic systems, nucleic acid or protein purification modules, arrays (e.g., nucleic acid arrays), detectors, thermocyclers or combinations thereof, e.g., for acquiring samples, diluting or aliquoting samples, purifying marker materials (e.g., nucleic acids or proteins), amplifying marker nucleic acids, detecting amplified marker nucleic acids, and the like.

For example, automated devices that can be incorporated into the systems herein have been used to assess a variety of biological phenomena, including, e.g., expression levels of genes in response to selected stimuli (Service (1998) “Microchips Arrays Put DNA on the Spot” Science 282:396-399), high throughput DNA genotyping (Zhang et al. (1999) “Automated and Integrated System for High-Throughput DNA Genotyping Directly from Blood” Anal. Chem. 71:1138-1145) and many others. Similarly, integrated systems for performing mixing experiments, DNA amplification, DNA sequencing and the like are also available. See, e.g., Service (1998) “Coming Soon: the Pocket DNA Sequencer” Science 282: 399-401. A variety of automated system components are available, e.g., from Caliper Technologies (Hopkinton, Mass.), which utilize various Zymate systems, which typically include, e.g., robotics and fluid handling modules. Similarly, the common ORCA® robot, which is used in a variety of laboratory systems, e.g., for microtiter tray manipulation, is also commercially available, e.g., from Beckman Coulter, Inc. (Fullerton, Calif.). Similarly, commercially available microfluidic devices that can be used as system components in the present invention include those from, e.g., Caliper Technologies. Furthermore, the patent and technical literature includes numerous examples of microfluidic systems, including those that can interface directly with microwell plates for automated fluid handling. These can be applied to the present invention, e.g., in the context of fluid handlers for performing cell-based assays to detect cell or tissue autonomous phenotypes.

Any of a variety of liquid handling and/or array configurations can be used in the systems herein, e.g., for performing cell or tissue response assays. One common format for use in the systems herein is a microtiter plate, in which the array or liquid handler includes a microtiter tray. Such trays are commercially available and can be ordered in a variety of well sizes and numbers of wells per tray, as well as with any of a variety of functionalized surfaces for binding of assay or array components. Common trays include the ubiquitous 96 well plate, with 384 and 1536 well plates also in common use. Samples can be processed in such trays, with all of the processing steps being performed in the trays. Samples can also be processed in microfluidic apparatus, or combinations of microtiter and microfluidic apparatus.

In addition to liquid phase arrays, components can be stored in or analyzed on solid phase arrays. These arrays fix materials in a spatially accessible pattern (e.g., a grid of rows and columns) onto a solid substrate such as a membrane (e.g., nylon or nitrocellulose), a polymer or ceramic surface, a glass or modified silica surface, a metal surface, or the like. Components can be accessed, e.g., by hybridization, by local rehydration (e.g., using a pipette or other fluid handling element) and fluidic transfer, or by scraping the array or cutting out sites of interest on the array.

The system can also include detection apparatus that is used to detect allele information, using any of the approached noted herein. For example, a detector configured to detect real-time PCR products (e.g., a light detector, such as a fluorescence detector) or an array reader can be incorporated into the system. For example, the detector can be configured to detect a light emission from a hybridization or amplification reaction comprising an allele of interest, wherein the light emission is indicative of the presence or absence of the allele. Optionally, an operable linkage between the detector and a computer that comprises the system instructions noted above is provided, allowing for automatic input of detected allele-specific information to the computer, which can, e.g., store the database information and/or execute the system instructions to compare the detected allele specific information to the look up table.

Probes that are used to generate information detected by the detector can also be incorporated within the system, along with any other hardware or software for using the probes to detect the amplicon. These can include thermocycler elements (e.g., for performing PCR or LCR amplification of the allele to be detected by the probes), arrays upon which the probes are arrayed and/or hybridized, or the like. The fluid handling elements noted above for processing samples, can be used for moving sample materials (e.g., template nucleic acids and/or proteins to be detected) primers, probes, amplicons, or the like into contact with one another. For example, the system can include a set of marker probes or primers configured to detect at least one allele of one or more genes or linked loci associated with a cell or tissue autonomous phenotype. The detector module is configured to detect one or more signal outputs from the set of marker probes or primers, or an amplicon produced from the set of marker probes or primers, thereby identifying the presence or absence of the allele.

The sample to be analyzed is optionally part of the system, or can be considered separate from it. The sample optionally includes e.g., genomic DNA, amplified genomic DNA, cDNA, amplified cDNA, RNA, amplified RNA, proteins, etc., as noted herein. In one aspect, the sample is derived from a mammal such as a human patient. In another, the sample is derived from an agricultural source.

Optionally, system components for interfacing with a user are provided. For example, the systems can include a user viewable display for viewing an output of computer-implemented system instructions, user input devices (e.g., keyboards or pointing devices such as a mouse) for inputting user commands and activating the system, etc. Typically, the system of interest includes a computer, wherein the various computer-implemented system instructions are embodied in computer software, e.g., stored on computer readable media.

Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Sequel™, Oracle™, Paradox™) can be adapted to the present invention by inputting a character string corresponding to an allele herein, or an association between an allele and a phenotype. For example, the systems can include software having the appropriate character string information, e.g., used in conjunction with a user interface (e.g., a GUI in a standard operating system such as a Windows, Macintosh or LINUX system) to manipulate strings of characters. Specialized sequence alignment programs such as BLAST can also be incorporated into the systems of the invention for alignment of nucleic acids or proteins (or corresponding character strings) e.g., for identifying and relating multiple alleles.

As noted, systems can include a computer with an appropriate database and an allele sequence or correlation of the invention. Software for aligning sequences, as well as data sets entered into the software system comprising any of the sequences herein can be a feature of the invention. The computer can be, e.g., a PC (Intel x86 or Pentium chip-compatible DOS™, OS2™ WINDOWS™ WINDOWS NT™, WINDOWS95™, WINDOWS98™, WINDOWS2000, WINDOWSME, or LINUX based machine, a MACINTOSH™, Power PC, or a UNIX based (e.g., SUN™ work station or LINUX based machine) or other commercially common computer which is known to one of skill. Software for entering and aligning or otherwise manipulating sequences is available, e.g., BLASTP and BLASTN, or can easily be constructed by one of skill using a standard programming language such as Visualbasic, Fortran, Basic, Java, or the like.

The following example is provided solely to illustrate the invention, and not to limit it. One of skill will recognize any of a variety of non-critical parameters that can be altered while achieving essentially similar results.

### Example Association Study for Radiation Sensitivity

The following is a description of an example association study between a cell-autonomous radiation sensitivity phenotype and genotype. Similar approaches are used for other cell autonomous phenotypes, e.g., as described throughout.

In this example, an association study is first performed to identify genetic loci that are involved in a cellular phenotype (e.g., radiation sensitivity). Cells (e.g., blood cells or skin fibroblasts) are collected from ˜1000 “normal” individuals and are used to produce cell lines for study. These cell lines are tested for radiation sensitivity and are genotyped at genetic loci across the genome (e.g., ˜300,000 loci). Loci that are associated with radiation sensitivity are identified (e.g., ˜100 loci). These loci are characterized with regards to their impact on the radiation sensitivity of the cell lines.

In order to test whether the genetic variation underlying fundamental normal cellular processes (e.g., response to radiation exposure) is predictive of the radiation sensitivity of an entire organism from which the cells are derived, data from a clinical trial is utilized. Individuals who are being treated with radiation (e.g., breast cancer patients) are examined to determine their sensitivity to the radiation treatment (“measured radiation sensitivity”). These individuals are further genotyped at the ˜100 loci identified in the association study, and based on the previously determined impact of the ˜100 loci on the radiation sensitivity of the cell lines tested, predictions are made regarding the radiation sensitivity of the patients (“predicted radiation sensitivity”). The predicted radiation sensitivities are then compared to the measured radiation sensitivities of the patients in order to determine whether the ˜100 loci previously found to be associated with radiation sensitivity in the cell lines are also associated with radiation sensitivity of an entire organism.

Those loci that are found to be predictive of radiation sensitivity of the organism may then be used to screen future patients as a means to ascertain their radiation sensitivity prior to radiation treatment, and to adjust their treatment (e.g., exposure levels) accordingly.

### Example for Deconstructing Complex Traits Into Endophenotypes

A challenge in the field of human genomics is to develop methodologies for rapid identification and functional characterization of DNA variants contributing to human phenotypic differences. Understanding the molecular mechanisms underlying bio-medically important human phenotypic differences, such as disease susceptibility and drug response, is useful in establishing the practice of personalized medicine to permit delivery of the most efficacious and safe drugs to the patient. Currently, the relationship between DNA variation and human phenotypic differences is poorly understood. For example, there is evidence that both common variants, defined as those present in 10% or more of the individuals around the world, and rare variants, defined as those present in only a limited number of individuals, contribute to the observed variation in complex human traits.

Genetic association studies are used to identify common variants associated with disease and drug response through large scale genotyping of SNP markers in matched case and control patient populations. The common-disease/common-variant hypothesis proposes that common variants occur in a wide variety of normal individuals and can result in complex phenotypic traits when present in specific combinations, although no single variant is either necessary or sufficient to cause the phenotype. A resource of common variants identified in a global population of normal individuals (see also, e.g., Hinds, et al. (2005) “Whole-genome patterns of common DNA variation in three human populations” Science 307, 1072-9) in combination with genetic association studies, provides a powerful approach for identifying common variants underlying complex human traits.

Numerous genetic association studies have been conducted and many have been published, describing correlations between one or a small number of SNPs with variation in a human trait; but most of these associations have proven difficult to replicate. Although association studies face a number of challenges that contribute to the difficulties of replicating findings, a large factor is likely due to the depth of complexity of the traits being analyzed. For example, a complex disease such as type II diabetes is not a uniform disease but a group of related metabolic disorders sharing a common phenotype: unregulated control of blood sugar levels (see also, Busch, & Hegele (2001). “Genetic determinants of type 2 diabetes mellitus” Clin Genet 60, 243-54). Type II diabetes can arise through contributions from a number of endophenotypes including: 1. increased insulin resistance in muscle and fat; 2. increased insulin resistance in the liver; 3. reduced insulin secretion from pancreatic β-cells; and 4. environmental factors such as lifestyle and body mass index.

These compound origins of type II diabetes make it difficult to identify SNP markers highly associated with the disease in standard genetic association studies. Different subsets of SNPs are likely to be associated with different mechanisms of the disease and the dilutive effect of the “mixed” patient population results in none of the individual SNPs being highly predictive of type II diabetes in the general population. For instance, if a particular SNP contributes up to 5% of the variation in insulin response in a particular tissue, and if insulin insensitivity in this tissue plays an important role in 30% of all cases of type II diabetes, the identified SNP would only contribute to 1-2% of the prevalence of type II diabetes overall. An effect of this size would be difficult to detect and/or replicate in standard population-based association studies and the SNP would likely have little predictive value. The present invention overcomes these difficulties, making it possible to analyze the DNA of an individual, e.g., with type II diabetes, to determine the affected underlying biological systems which result in the insulin resistance, such that the most effective treatment can be administered.

In vitro Approach for Studying Endophenotypes

Genetically well-characterized human lymphoblastoid and embryonic stem (ES) cell lines are used in this example to identify and functionally characterize DNA variants underlying endophenotypes contributing to the variability of drug response and disease susceptibility phenotypes in humans. This approach is based on the assumption that complex traits can be deconstructed into endophenotypes, which represent simpler biological systems, allowing for more straightforward and successful genetic analysis. A series of hundreds of lymphoblastoid and ES cell lines are obtained and genotyped for ˜250,000 highly-informative SNP markers. Once each cell line has been genotyped, laboratory studies are performed using the cell lines to investigate multiple complex phenotypes relevant to biomedically-important traits. Thus, the genetic information is used to find DNA variants associated with a variety of phenotypes, allowing many important questions to be addressed from this one data set.

Lymphoblastoid cell lines have proven to be useful in many studies for evaluation of clinically relevant complex traits (Monks, et al. (2004) “Genetic inheritance of gene expression in human cell lines.” Am J Hum Genet 75, 1094-105; Guillamet et al. (2004) “In vitro DNA damage by arsenic compounds in a human lymphoblastoid cell line (TK6) assessed by the alkaline Comet assay.” Mutagenesis 19, 129-35). The studies of this example are initiated by assessing the variability and heritability of selected traits in 90 publicly available lymphoblastoid cell lines from 30 family trios (CEPH; on the world wide web at www(dot)cephb(dot)fr/). The first traits to be investigated are variations in lymphoblastoid cell line susceptibility to chemotherapeutic agents (see e.g., Watters, et al. (2004) “Genome-wide discovery of loci influencing chemotherapy cytotoxicity.” Proc Natl Acad Sci USA 101, 11809-14 for a description of appropriate assays), radiation (see, e.g., Correa & Cheung (2004) “Genetic variation in radiation-induced expression phenotypes” Am J Hum Genet 75, 885-90 for a description of relevant assays) and other challenges by assessment of cell culture growth rate, cell morphology, and rate of thymidine incorporation as a measure of DNA synthesis.

Phenotypic responses that exhibit significant variability and heritability are then assessed for genetic association using a collection of hundreds of human lymphoblastoid cell lines including 270 cell lines that have been extensively genotyped as part of the HapMap Project (on the world wide web at hapmap(dot)org/), and additional cell lines that we will genotype for at least 250,000 highly-informative SNP markers. The thorough genetic characterization of these cell lines provides unprecedented power to associate specific genetic loci with phenotypic traits of interest.

The pluripotent potential of ES cells to differentiate into a large variety of tissues allows for the investigation of tissue-specific phenomena that cannot be addressed in lymphoblastoid cell lines or individual patients, such as the actions of cardioactive drugs and neurotransmitters on ES cell derived cardiomyocytes (see, e.g., He et al. (2003) “Human embryonic stem cells develop into multiple types of cardiac myocytes: action potential characterization.” Circ Res 93, 32-9 for a description of relevant assays), and dopaminergic neurons (see e.g., Martinat et al. (2004) “Sensitivity to oxidative stress in DJ-1-deficient dopamine neurons: an ES-derived cell model of primary parkinsonism.” PLoS Biol 2, e327), respectively. ES cell lines currently approved for federal research can be used in feasibility and development studies; subsequent investigations of hundreds or even thousands of additional cell lines can also be conducted. There are currently 78 ES cell lines approved for federal research, of which 22 are available. As additional federally-approved ES cell lines become available, these can also be incorporated into the above studies.

Once SNP markers associated with variability in drug response and/or susceptibility to disease are identified, the lymphoblastoid and ES cell lines are used to make detailed investigations into the molecular mechanisms underlying the associations, and thus gain a better understanding of the biology underpinning the observed phenotypes. Standard functional analyses are used to characterize the properties of protein coding and regulatory SNPs, such as comparative genomics, protein binding studies, transient expression studies, and expression analysis. See also, Hinds et al. (2005) “Whole-genome patterns of common DNA variation in three human populations. Science” 307, 1072-9, and Patil et al. “Blocks of limited haplotype diversity revealed by high-resolution scanning of human chromosome 21.” Science 294, 1719-23. The publication of extensive genotypes for lymphoblastoid and ES cell lines, along with many of the phenotypes and genetic associations observed, are useful tools for both academic and industrial research groups around the world. Understanding the genetics and molecular mechanisms that contribute to variation in drug response and susceptibility to diseases in humans is made difficult by the interplay between the multiple biological systems involved in each complex trait. The use of lymphoblastoid and ES cell-lines allows the study of endophenotypes, reducing the complexity of the traits investigated and increasing the possibility of identifying SNPs that have predictive value, and thus bio-medical relevance for personalized medicine.

Large numbers of genetically well-characterized cell-lines are used to identify associations between DNA variants and complex traits; these same cell-lines are also used to identify and characterize the molecular mechanisms involved.

Although the above discussion has presented the present invention according to specific methods, systems, compositions and apparatus, the present invention has a broader range of applicability. Further, while the foregoing invention has been described in some detail for purposes of clarity and understanding, it will be clear to one skilled in the art from a reading of this disclosure that various changes in form and detail can be made without departing from the true scope of the invention. For example, all the methods, techniques, systems, devices, kits, apparatus, etc., described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of correlating a cell or tissue autonomous phenotype to a genotype, the method comprising:
detecting variance in a cell or tissue autonomous phenotype in a population of cells or tissues;
accessing genotype information for the cells or tissues in the population; and,
correlating the variance to the genotype information, thereby correlating the cell or tissue autonomous phenotype and the genotype.

2. The method of claim 1, wherein the cell or tissue autonomous phenotype is a cellular response to a stimulus.

3. The method of claim 1, wherein the cell or tissue autonomous phenotype is selected from the group consisting of: a radiation response of the cell or tissue; a response of the cell or tissue to an anti-cancer drug; a response of the cell or tissue to a therapeutic agent; a measure of DNA damage repair by the cell or tissue; an immune response by the cell or tissue; an anti-inflammatory response of the cell or tissue; cytokine production by the cell or tissue; energy metabolism of the cell or tissue; oxygen consumption of the cell or tissue; a rate of DNA replication, transcription or translation; an insulin sensitivity or resistance of muscle, fat, or liver cells or tissues; a level of insulin secretion from pancreatic β cells; cell migration; an electrical response of the cell or tissue; a flow of ions across a membrane of the cell or tissue; apoptosis of the cell or tissue; an expression level of a housekeeping gene of the cell or tissue; an activity of a hosekeeping gene product; and cell cycle regulation of the cell or tissue.

4. The method of claim 1, wherein the cell or tissue autonomous phenotype is correlated with a physiological status of an individual from which a cell or tissue having the cell or tissue autonomous phenotype is derived.

5. The method of claim 1, wherein the detecting comprises detecting natural or systematic variation of the cell or tissue autonomous phenotype in the population of cells or tissues.

6. The method of claim 1, wherein the detecting comprises detecting high and low responder cells or tissues in the population, wherein the high responder cells or tissues have a higher response than the low responder cells or tissues to a selected stimulus.

7. The method of claim 6, wherein the high responder cells or tissues display a higher enzymatic response to a stimulus than the low responder cells or tissues.

8. The method of claim 6, wherein the high responder cells or tissues are selected from the highest 25% of the population of cells or tissues in the response to the selected stimulus and the low responder cells or tissues are selected from the lowest 25% of the population of cells or tissues in the response to the selected stimulus.

9. The method of claim 1, wherein the population of cells or tissues comprises an in vitro population of primary or cultured cells or tissues derived from a population of patients.

10. The method of claim 9, wherein the cells or tissues comprise stem cells.

11. The method of claim 9, wherein the cells or tissues are differentiated or dedifferentiated in vitro.

12. The method of claim 9, wherein the cells of the population are individual cells.

13. The method of claim 1, wherein the cells or tissues are from a cell or tissue bank, wherein the cells or tissues of the bank are genotyped.

14. The method of claim 13, wherein the bank has about 1,000 or more different genotyped cell or tissue lines.

15. The method of claim 1, wherein the population of cells or tissues comprises a population of tissue samples derived from a population of patients.

16. The method of claim 1, wherein the cells are constituted within tissues or cell aggregates.

17. The method of claim 16, wherein the tissues are whole tissues.

18. The method of claim 1, wherein said detecting variance comprises separating the population of cells or tissues into case and control sets based upon the variance of the cell or tissue autonomous phenotype in the population.

19. The method of claim 18, wherein the population of cells or tissues comprises positive or negative control cells or tissues for the case set.

20. The method of claim 19, wherein the positive control cells or tissues and the case control set of cells or tissues are derived from common cell lines or tissues or cell or tissue types and the negative control cells or tissues and the control set of cells or tissues are derived from common cell lines or cell types.

21. The method of claim 1, wherein the cell or tissue autonomous phenotype is detected a plurality of times for each cell or tissue type within the population of cells or tissues, thereby amplifying correlation certainty during said correlating.

22. The method of claim 21, wherein the plurality of times comprises 100 or more detections of the phenotype for each cell or tissue type.

23. The method of claim 1, wherein accessing the genotype information comprises identifying polymorphisms or a genotype in members of the population of cells or tissues.

24. The method of claim 23, wherein the polymorphisms are identified after said detecting.

25. The method of claim 23, wherein the genotype information is accessed before said detecting.

26. The method of claim 23, wherein the genotype comprises more than about 100,000 SNPs.

27. The method of claim 23, wherein the genotype comprises about 250,000 SNPs or more.

28. The method of claim 23, wherein the genotype comprises a genome-wide sample of polymorphisms.

29. The method of claim 23, wherein at least a portion of the polymorphisms are pre-selected to have an effect on, a predicted effect on, a correlation to or a predicted correlation to the cell autonomous phenotype.

30. The method of claim 23, wherein a portion of the polymorphisms display 10% or less variation in a patient population.

31. The method of claim 1, wherein said correlating comprises determining a genetic bar code for the cell or tissue autonomous phenotype.

32. The method of claim 1, further comprising determining or predicting a correlation between the genotype or cell autonomous phenotype and a patient condition.

33. The method of claim 32, wherein the patient condition is selected from the group consisting of: a side effect, a side effect predisposition, a disease state, a disease predisposition, a disease prognosis, a disease treatment response and a disease treatment efficacy.

34. The method of claim 1, further comprising diagnosing, detecting, detecting a predisposition for, predicting an outcome of and/or selecting a treatment regimen for a disease in a patient base upon a correlation between a cell autonomous phenotype of the patient and the genotype.

35. A method diagnosing, detecting, detecting a predisposition for, predicting an outcome of and/or selecting a treatment regimen for a disease, the method comprising:
detecting at least one in vitro cell or tissue autonomous phenotype of a cell or tissue from a patient and/or detecting a genotype correlated to the cell autonomous phenotype; and,
accessing a database comprising a correlation between the cell or tissue autonomous phenotype and/or genotype and one or more of: a side effect, a side effect predisposition, a disease, a predisposition to a disease, a prognosis of a disease, and/or treatment response or efficacy for a disease; and,
based on the correlation: diagnosing, detecting, detecting a predisposition for, predicting an outcome of and/or selecting a treatment regimen for the disease.

36. The method of claim 35, wherein the cell or tissue autonomous phenotype is an indicator for the phsiological status of the patient.

37. The method of claim 35, wherein the cell or tissue autonomous phenotype is selected from the group consisting of: a radiation response of the cell or tissue; a response of the cell or tissue to an anti-cancer drug; a response of the cell or tissue to a therapeutic agent; a measure of DNA damage repair by the cell or tissue; an immune response by the cell or tissue; an anti-inflammatory response of the cell or tissue; cytokine production by the cell or tissue; energy metabolism of the cell or tissue; oxygen consumption of the cell or tissue; a rate of DNA replication, transcription or translation; an insulin sensitivity or resistance of muscle, fat, or liver cells or tissues; a level of insulin secretion from pancreatic β cells; cell migration; an electrical response of the cell or tissue; a flow of ions across a membrane of the cell or tissue; apoptosis of the cell or tissue; an expression level of a housekeeping gene of the cell or tissue; an activity of a hosekeeping gene product; and cell cycle regulation of the cell or tissue.

38. The method of claim 35, wherein the disease is selected from the group consisting of: cancer, an infectious disease, a viral infection, a bacterial infection, an immune disorder, an autoimmune disorder, obesity, diabetes, cardiovascular disease, a metabolic disorder, metabolic syndrome, a neurodegnerative disease, a CNS disorder, a transplant-related condition, and a genetic disease.

39. The method of claim 35, wherein the treatment regimen is selected from the group consisting of: surgery, exposure to radiation, administration of a drug, administration of an anti-cancer drug, administration of an anti-viral drug, administration of an antibiotic, administration of an immune suppressor or enhancer, administration of a cardiovascular drug, administration of a cholosterol level regulating drug, administration of a neurological drug, administration of an anti-rejection drug, administration of an enzyme inhibitor, administration of an enzyme activator, diet, and exercize.

40. The method of claim 35, wherein the cell from the patient is taken directly from the patient.

41. The method of claim 35, wherein the cell from the patient is derived from the patient by culture, differentiation and/or dedifferentiation.

42. The method of claim 35, wherein the cell autonomous phenotype is verified in replicate experiments using a plurality of cells from the patient.

43. The method of claim 35, wherein detecting the genotype comprises detecting a set of selected polymorphisms that correlate, positively or negatively, to the cell autonomous phenotype.

44. The method of claim 43, wherein the set of selected polymorphisms comprises fewer than 100 polymorphisms.

45. The method of claim 35, wherein detecting the genotype comprises detecting a set of selected polymorphisms that correlate, positively or negatively, to said disease, said predisposition to the disease, said prognosis of the disease, and/or said treatment response or efficacy for the disease.

46. The method of claim 35, wherein the database comprises a lookup table that comprises correlation relationships for the cell autonomous phenotype and/or genotype and the side effect, the side effect predisposition, the disease, the predisposition to the disease, the prognosis of the disease, and/or the treatment efficacy for the disease.

47. The method of claim 35, wherein the database is a heuristic database that refines the correlation between genotype, cell autonomous phenotype, and/or the disease, predisposition to the disease, prognosis of the disease, and/or treatment response or efficacy for the disease, based upon inputs regarding the correlation.

48. The method of claim 47, wherein the heuristic database comprises one or more of: a neural network (NN), a statistical model (SM), a hidden Markov model (HMM), principal component analysis (PCA), classification and regression trees (CART), multivariate adaptive regression splines (MARS), genetic algorithms (GA), multiple linear regression (MLR), variable importance for projection (VIP), inverse least squares (ILS), and partial least square (PLS).
